(19) United States
(12) Patent Application Publication
(10) Pub. No.: US 2024/0391880 A1
(43) Pub. Date: Nov. 28, 2024

(54) KCa3.1 INHIBITORS FOR PODOCYTE PROTECTION

(71) Applicants: Icahn School of Medicine at Mount Sinai, New York, NY (US); Long Island University, Brookville, NY (US)

(72) Inventors: Kirk CAMPBELL, New York, NY (US); Bhaskar DAS, Brookville, NY (US); Jenny WONG, New York, NY (US)

(73) Assignees: Icahn School of Medicine at Mount Sinai, New York, NY (US); Long Island University, Brookville, NY (US)

(21) Appl. No.: 18/691,500

(22) PCT Filed: Sep. 14, 2022
(86) PCT No.: PCT/US2022/076389

§ 371 Date: Mar. 13, 2024

Related U.S. Application Data
(60) Provisional application No. 63/244,656, filed on Sep. 15, 2021.

(51) Int. Cl. C07D 235/18; A61K 31/4184; A61K 31/437; A61P 13/12; C07D 471/04

(52) U.S. Cl. CPC C07D 235/18; A61K 31/4184; A61K 31/437; A61P 13/12; C07D 471/04

(57) ABSTRACT

The present disclosure relates to compounds of Formula I and II and pharmaceutically acceptable salts thereof:

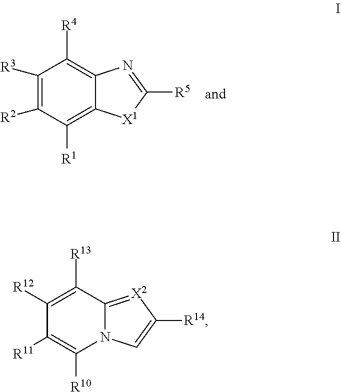

and a pharmaceutical composition including a compound of formula I or II and a pharmaceutically acceptable excipient. Also provided is a method of administering a compound of formula I or II or a physiological salt thereof, or said pharmaceutical composition to a subject, wherein the subject suffers from kidney disease or is at risk of kidney disease.

### CROSS-REFERENCE TO RELATED APPLICATIONS

This application claims priority from U.S. Provisional Application No. 63/244,656, filed on Sep. 15, 2021, which is incorporated herein by reference in its entirety.

### GOVERNMENT RIGHTS STATEMENT

This invention was made with government support under grant numbers DK103022 and DK122807 awarded by the National Institutes of Health. The government has certain rights in the invention.

### FIELD

The present disclosure relates generally to compounds that inhibit KCa3.1 and are therefore useful for treating kidney diseases. The compounds are generally benozimidazole and imidazopyridine derivatives.

### BACKGROUND

Kidney podocytes are a target cell for injury across the spectrum of proteinuric disease irrespective of etiology (1-3). Primary and secondary podocytopathies together account for 90% of all end stage kidney disease in the U.S. at an annual expenditure of approximately $20 billion per year (4). There is emerging evidence of a threshold podocyte number below which proteinuria and glomerulosclerosis become irreversible (5). Numerous reports have implicated increased intracellular calcium in podocyte injury and the pathogenesis of glomerular disease (6, 7), though the underlying mechanisms remain poorly defined. There is a need for a better understanding of mechanisms underlying podocyte survival, particularly under disease conditions. Additionally, there are no podocyte specific drugs approved for clinical use (8, 9).

The Hippo pathway is a conserved kinase cascade from Drosophila to mammals and regulates organ size and cell survival (10, 11). The effector and Hippo kinase (LATS and MST) target Yes associated protein (YAP) has been extensively studied in the oncology field as a chemotherapeutic drug development target due to its role as a potent oncogene (12, 13). The Hippo pathway plays a role in podocyte survival and glomerular disease progression (14-19). Podocyte-specific YAP silencing causes focal segmental glomerulosclerosis (FSGS) (15). Patients with biopsy-proven FSGS also have decreased glomerular YAP expression. Furthermore, the tyrosine kinase inhibitor dasatinib, used clinically to treat philadelphia chromosome positive acute lymphoblastic leukemia and chronic myeloid leukemia, causes YAP inhibition, albuminuria and glomerular disease (20).

### SUMMARY

The present disclosure is directed to compounds, pharmaceutical compositions, and methods for inhibiting KCa3.1 and thereby treating various kidney diseases.

In an aspect, the disclosure relates to a compound of formula I:

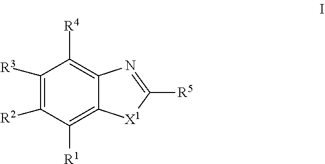

or a pharmaceutically acceptable salt thereof, wherein
X¹ is selected from N, O, and S;
R¹, R², R³, and R⁴ are each independently selected from hydrogen, halogen, CN, C(═O)N(R⁶)₂, C(═O)OR⁶, (C₃-C₁₀)carbocycle, heterocycle, and B(OH)₂;
R⁵ is selected from (C₃-C₁₀)carbocycle and heterocycle, wherein each is optionally substituted with one or more of halogen, CN, C(=O)N(R⁶)₂, C(═O)OR⁶, (C₃-C₁₀)carbocycle, heterocycle, or B(OH)₂; and
R⁶ in each occurrence is independently selected from hydrogen and (C₁-C₆)alkyl.

In another aspect, the disclosure relates to a compound of formula II:

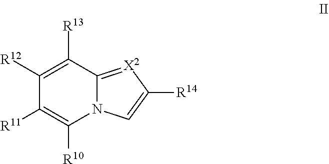

or a pharmaceutically acceptable salt thereof, wherein
X² is selected from N, O, and S;
R¹⁰, R¹¹, R¹², and R¹³ are each independently selected from hydrogen, halogen, CN, C(═O)N(R¹⁵)₂, C(═O)OR¹⁵, C(═O)R¹⁵, —N(R¹⁵)₂, (C₁-C₆)alkoxy, (C₁-C₆)thiaalkyl, (C₁-C₆)oxaalkyl, oxo(C₁-C₆)azaalkene, (C₃-C₁₀)carbocycle, heterocycle, and B(OH)₂, wherein said heterocycle is optionally substituted with one or more of —N(R¹⁵)₂, wherein R¹⁵ in each occurrence is independently selected from hydrogen and (C₁-C₆)alkyl; and
R¹⁴ is selected from (C₃-C₁₀)carbocycle and heterocycle, wherein each is optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂.

In another aspect, disclosed is a pharmaceutical composition including a compound, or a pharmaceutically acceptable salt thereof, as described herein, and a pharmaceutically acceptable excipient.

In another aspect, provided is a method of treatment, including administering a compound, or a pharmaceutically acceptable salt thereof, as described herein, to a subject, wherein the subject suffers from kidney disease or is at risk of kidney disease.

In another aspect, provided is a method of treatment, including administering a pharmaceutical composition, as described herein, to a subject, wherein the subject suffers from kidney disease or is at risk of kidney disease.

### BRIEF DESCRIPTION OF THE DRAWINGS

These and other features, aspects, and advantages of the present disclosure will become better understood when the following detailed description is read with reference to the accompanying drawings, wherein:

FIGS. 1A-1D depict the disruption of podocyte actin cytoskeletal integrity by YAP silencing; FIG. 1A is an immunofluorescence labeling of paxillin in control and YAP shRNA (YAP KD) immortalized mouse podocytes; paxillin (green) showing focal adhesions, phalloidin (red) showing stress fibers, and nuclear expression staining with DAPI (blue); reduction in cell size, focal adhesion, and decrease in stress fibers with YAP knockdown; FIG. 1B is a heatmap and histogram comparing the transcriptomic profile of control and YAP knockdown podocytes; FIG. 1C depicts a KEGG enrichment analysis; and FIG. 1D depicts a Wikipathway enrichment analysis;

FIGS. 2A-2E depict increases in KCNN4 gene and encoded KCa3.1 expression by YAP silencing; FIG. 2A is a network analysis of differentially expressed genes, wherein the node with the highest centrality and connectivity is actin; FIG. 2B is a manually curated “potassium channel regulator activity” ontology; FIG. 2C is a real-time PCR showing increased KCNN4 gene expression in YAP KD podocytes; FIG. 2D is an immunofluorescence staining showing increased KCa3.1 expression in YAP KD podocytes; and FIG. 2E is ChIP-Seq showing YAP localization on multiple sites near the KCNN4 gene in podocytes;

FIGS. 3A-3C depict the correlation of KCNN4 glomerular gene expression with clinical outcomes; FIG. 3A shows KCa3.1 expression is increased in biopsies of patients with FSGS; FIG. 3B shows that increased glomerular KCNN4 gene expression in FSGS patients from the NEPTUNE cohort is associated with more rapid decline in eGFR; and FIG. 3C shows that KCNN4 overexpression causes actin cytoskeletal disruption in immortalized mouse podocytes;

FIGS. 4A-4D depict the synthesis of novel KCa3.1 inhibitors; FIG. 4A shows the chemical structures of novel TRAM34 analogs BT562-566; FIG. 4B shows pore and allosteric binding pockets of KCa3.1; FIG. 4C shows BT563 inside the binding pocket with a 2D ligand interaction diagram highlighting the major intermolecular interactions with the pocket residues; and FIG. 4D shows BT564 inside the binding pocket with a 2D ligand interaction diagram highlighting the major intermolecular interactions with the pocket residues;

FIG. 5 depicts KCa3.1 analog effects on YAP KD podocyte morphology; BT563 and BT564 most consistently rescued podocyte morphologic changes conferred by YAP silencing; phalloidin (red) and paxillin (in green);

FIGS. 6A-6H depict the effect of KCa3.1 antagonists BT564 and BT563 on the current-voltage (I-V) relationship for YAP KD and KCNN4 overexpressing podocyte lines with black line indicating the mean pre-drug I-V plot, red line indicating the mean I-V plots from the same cells in presence of the drug (after a 2-5 minute incubation), X axes representing the applied voltage across the cell membrane, Y axes representing the resulting currents in response to the applied voltages, and blue arrows indicating the calculated equilibrium potential (−83 mV) for K+ (VK);

FIGS. 6I and 6J depict the effect of KCa3.1 antagonist TRAM 34 in presence of BK channel antagonist paxilline on current-voltage relationship of (FIG. 6I) YAP knockout and (FIG. 6J) KCNN overexpressing cell lines;

FIGS. 6K and 6L depict the effect of nonselective potassium channel blocker TEA (tetraethylammonium) on I-V relationship of (FIG. 6K) YAP knockdown and (FIG. 6L) KCNN4 overexpressing cell lines;

FIGS. 7A-7C depict the effect of the KCa3.1 antagonists on intracellular calcium dynamics; FIG. 7A shows the time to peak of calcium uptake; FIG. 7B shows the time to CaD50; and FIG. 7C shows the time from stimuli to peak;

FIG. 8 depicts KCa3.1 inhibitors protecting podocytes from LPS-induced injury; and

FIGS. 9A-9C depict the effect of KCa3.1 inhibitors in LPS-induced glomerular disease; FIGS. 9A and 9B show that BT563 but not BT564 decreased albuminuria in LPS glomerulopathy; and FIG. 9C is a scanning EM showing BT563 rescue of LPS-induced podocyte foot process disruption.

### DETAILED DESCRIPTION

The present disclosure relates to compounds that may inhibit KCa3.1, and thereby treat kidney diseases. In an aspect, disclosed is a compound of formula I:

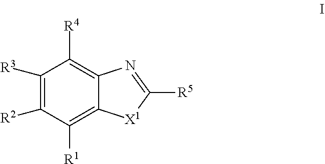

or a pharmaceutically acceptable salt thereof, wherein
X¹ is selected from N, O, and S;
R¹, R², R³, and R⁴ are each independently selected from hydrogen, halogen, CN, C(═O)N(R⁶)₂, C(═O)OR⁶, (C₃-C₁₀)carbocycle, heterocycle, and B(OH)₂;
R⁵ is selected from (C₃-C₁₀)carbocycle and heterocycle, wherein each is optionally substituted with one or more of halogen, CN, C(═O)N(R⁶)₂, C(═O)OR⁶, (C₃-C₁₀)carbocycle, heterocycle, or B(OH)₂; and
R⁶ in each occurrence is independently selected from hydrogen and (C₁-C₆)alkyl.

In an example, X¹ is N. In another example, X¹ is O. In yet another example, X¹ is S.

In an example, R¹ is hydrogen. In another example, R¹ is halogen. Non-limiting examples of halogen include Cl, Br, and I. In yet another example, R¹ is CN. In a further example, R¹ is C(═O)N(R⁶)₂. In yet a further example, R¹ is C(═O)OR⁶. In still a further example, R¹ is (C₃-C₁₀)carbocycle. In another further example, R¹ is heterocycle. In yet another further example, R¹ is B(OH)₂.

In an example, R² is hydrogen. In another example, R² is halogen. In yet another example, R² is CN. In a further example, R² is C(═O)N(R⁶)₂. In yet a further example, R² is C(═O)OR⁶. In still a further example, R² is (C₃-C₁₀)carbocycle. In another further example, R² is heterocycle. In yet another further example, R² is B(OH)₂.

In an example, R³ is hydrogen. In another example, R³ is halogen. In yet another example, R³ is CN. In a further example, R³ is C(═O)N(R⁶)₂. In yet a further example, R³ is C(═O)OR⁶. In still a further example, R³ is (C₃-C₁₀)carbocycle. In another further example, R³ is heterocycle. In yet another further example, R³ is B(OH)₂.

In an example, R⁴ is hydrogen. In another example, R⁴ is halogen. In yet another example, R+is CN. In a further example, R+is C(═O)N(R⁶)₂. In yet a further example, R⁴ is C(═O)OR⁶. In still a further example, R⁴ is (C₃-C₁₀)carbocycle. In another further example, R⁴ is heterocycle. In yet another further example, R+is B(OH)₂.

In an example, R⁵ is (C₃-C₁₀)carbocycle, wherein (C₃-C₁₀)carbocycle is optionally substituted with one or more of halogen, CN, C(═O)N(R⁶)₂, C(═O)OR⁶, (C₃-C₁₀)carbocycle, heterocycle, or B(OH)₂. In another example, R⁵ is heterocycle, wherein heterocycle is optionally substituted with one or more of halogen, CN, C(=O)N(R⁶)₂, C(═O)OR⁶, (C₃-C₁₀)carbocycle, heterocycle, or B(OH)₂. In yet another example, R⁵ may be pyridine, pyrimidine, pyrrole, thiophene, furan, or oxadiazol, wherein the pyridine, pyrimidine, pyrrole, thiophene, furan, and oxadiazol may be optionally substituted with one or more of halogen, CN, C(═O)N(R⁶)₂, C(═O)OR⁶, (C₃-C₁₀)carbocycle, heterocycle, or B(OH)₂.

In an example, R⁵ is R⁵ is

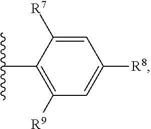

and [symbol] is the point of attachment.

In an example, R⁶ is hydrogen. In another example, R⁶ is (C₁-C₆)alkyl.

In an example, R⁷ is hydrogen. In another example, R⁷ is halogen. In yet another example, R⁷ is CN. In a further example, R⁷ is C(═O)N(R⁶)₂. In yet a further example, R⁷ is C(═O)OR⁶. In still a further example, R⁷ is (C₃-C₁₀)carbocycle. In still a further example, R⁷ is heterocycle, wherein the heterocycle is optionally substituted with one or more of (C₁-C₆)alkyl, halogen, CN, C(═O)N(R⁶)₂, C(═O)OR⁶, (C₃-C₁₀)carbocycle, heterocycle, or B(OH)₂. In another further example, R⁷ is B(OH)₂.

In an example, R⁸ is hydrogen. In another example, R⁸ is halogen. In yet another example, R⁸ is CN. In a further example, R⁸ is C(═O)N(R⁶)₂. In yet a further example, R⁸ is C(═O)OR⁶. In still a further example, R⁸ is (C₃-C₁₀)carbocycle. In still a further example, R⁸ is heterocycle, wherein the heterocycle is optionally substituted with one or more of (C₁-C₆)alkyl, halogen, CN, C(═O)N(R⁶)₂, C(═O)OR⁶, (C₃-C₁₀)carbocycle, heterocycle, or B(OH)₂. In another further example, R⁸ is B(OH)₂.

In an example, R⁹ is hydrogen. In another example, R⁹ is halogen. In yet another example, R⁹ is CN. In a further example, R⁹ is C(═O)N(R⁶)₂. In yet a further example, R⁹ is C(═O)OR⁶. In still a further example, R⁹ is (C₃-C₁₀)carbocycle. In still a further example, R⁹ is heterocycle, wherein the heterocycle is optionally substituted with one or more of (C₁-C₆)alkyl, halogen, CN, C(═O)N(R⁶)₂, C(═O)OR⁶, (C₃-C₁₀)carbocycle, heterocycle, or B(OH)₂. In another further example, R⁹ is B(OH)₂.

In an example, the compound of formula I, or a pharmaceutically acceptable salt thereof, may have the following structure:

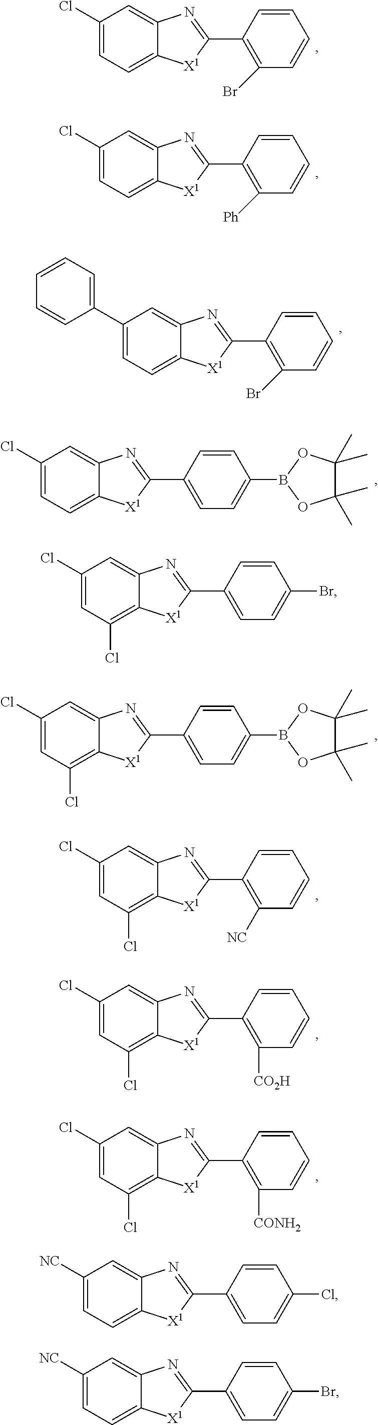

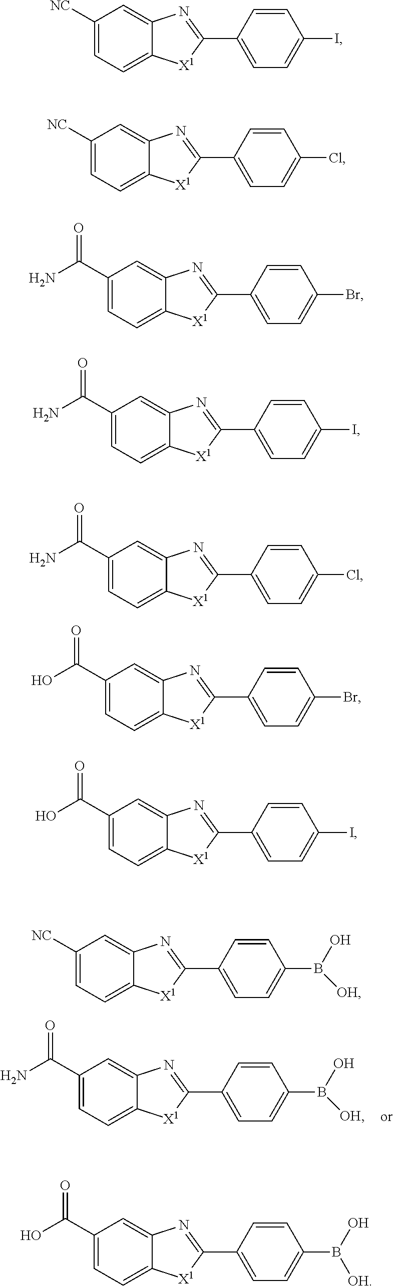

In another example, the compound of formula I, or a pharmaceutically acceptable salt thereof, may have the following structure:

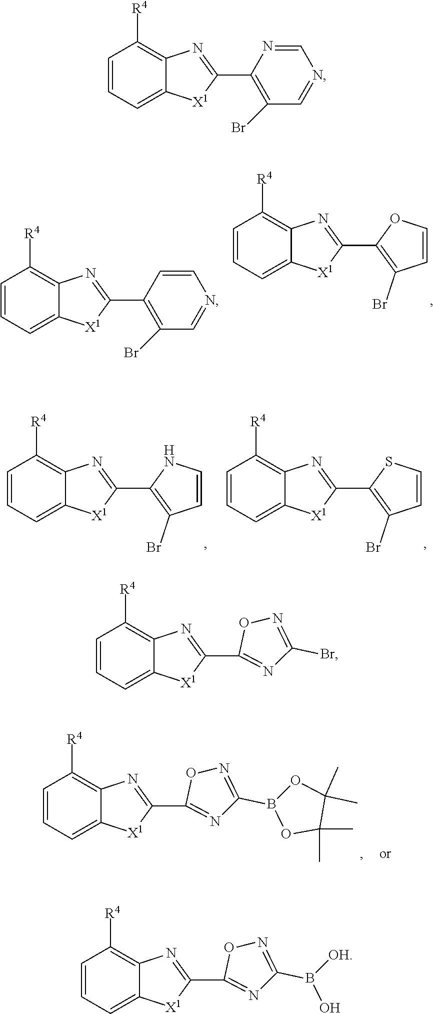

In yet another example, the compound of formula I, or a pharmaceutically acceptable salt thereof, may have the following structure:

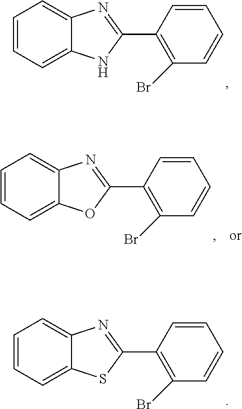 [SMILES: Brc1ccccc1-c1nc2ccccc2[nH]1 ; Brc1ccccc1-c1nc2ccccc2o1 ; Brc1ccccc1-c1nc2ccccc2s1]

In another aspect, disclosed is a compound of formula II:

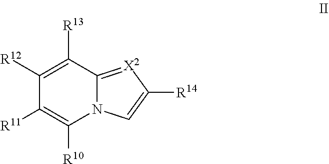

or a pharmaceutically acceptable salt thereof, wherein
X² is selected from N, O, and S;
R¹⁰, R¹¹, R¹², and R¹³ are each independently selected from hydrogen, halogen, CN, C(═O)N(R¹⁵)₂, C(═O)OR¹⁵, C(═O)R¹⁵,—N(R¹⁵)₂, (C₁-C₆)alkoxy, (C₁-C₆)thiaalkyl, (C₁-C₆)oxaalkyl, oxo(C₁-C₆)azaalkene, (C₃-C₁₀)carbocycle, heterocycle, and B(OH)₂, wherein said heterocycle is optionally substituted with one or more of —N(R¹⁵)₂, wherein R¹⁵ in each occurrence is independently selected from hydrogen and (C₁-C₆)alkyl; and
R¹⁴ is selected from (C₃-C₁₀)carbocycle and heterocycle, wherein each is optionally substituted with one or more of halogen, CN, C(═O)N(R¹⁵) 2, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂.

In an example, X² is N. In another example, X² is O. In yet another example, X² is S.

In an example, R¹⁰ is heterocycle optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂.

In another example, R¹¹ is heterocycle optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂.

In yet another example, R¹² is heterocycle optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂.

In still another example, R¹³ is heterocycle optionally substituted with one or more of halogen, CN, C(═O)N(R¹⁵)₂, C(═O)OR¹⁵, C(═O)R¹⁵,(C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂.

In an example, R¹⁴ is (C₃-C₁₀)carbocycle optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N (R¹⁵) 2. In another example, R¹⁴ is heterocycle optionally substituted with one or more of halogen, CN, C(═O)N(R¹⁵)₂, C(═O)OR¹⁵, C(═O)R¹⁵,(C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂.

In an example, R¹⁵ is hydrogen. In another example, R¹⁵ is (C₁-C₆)alkyl.

In yet another example, R¹⁴ is

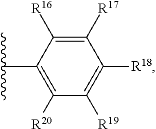

and [symbol] is the point of attachment.

In an example, R¹⁶ is hydrogen. In another example, R¹⁶ is halogen. In yet another example, R¹⁶ is CN. In still another example, R¹⁶ is C(═O)N(R¹⁵)₂, In a further example, R¹⁶ is C(═O)OR¹⁵. In yet a further example, R¹⁶ is C(═O)R¹⁵. In still a further example, R¹⁶ is —N(R¹⁵)₂. In still a further example, R¹⁶ is (C₁-C₆)alkoxy. In another further example, R¹⁶ is (C₁-C₆)thiaalkyl. In yet another further example, R¹⁶ is (C₁-C₆)oxaalkyl. In still another further example, R¹⁶ is oxo(C₁-C₆)azaalkene. In an example, R¹⁶ is (C₃-C₁₀)carbocycle. In another example, R¹⁶ is heterocycle is optionally substituted with one or more of —N(R¹⁵)₂. In yet another example, R¹⁶ is B(OH)₂.

In an example, R¹⁷ is hydrogen. In another example, R¹⁷ is halogen. In yet another example, R¹⁷ is CN. In still another example, R¹⁷ is C(═O)N(R¹⁵)₂, In a further example, R¹⁷ is C(═O)OR¹⁵. In yet a further example, R¹⁷ is C(═O)R¹⁵. In still a further example, R¹⁷ is —N(R¹⁵)₂. In still a further example, R¹⁷ is (C₁-C₆)alkoxy. In another further example, R¹⁷ is (C₁-C₆)thiaalkyl. In yet another further example, R¹⁷ is (C₁-C₆)oxaalkyl. In still another further example, R¹⁷ is oxo(C₁-C₆)azaalkene. In an example, R¹⁷ is (C₃-C₁₀)carbocycle. In another example, R¹⁷ is heterocycle is optionally substituted with one or more of —N(R¹⁵)₂. In yet another example, R¹⁷ is B(OH)₂.

In an example, R¹⁸ is hydrogen. In another example, R¹⁸ is halogen. In yet another example, R¹⁸ is CN. In still another example, R¹⁸ is C(═O)N(R¹⁵)₂, In a further example, R¹⁸ is C(═O)OR¹⁵. In yet a further example, R¹⁸ is C(═O)R¹⁵. In still a further example, R¹⁸ is —N(R¹⁵)₂. In still a further example, R¹⁸ is (C₁-C₆)alkoxy. In another further example, R¹⁸ is (C₁-C₆)thiaalkyl. In yet another further example, R¹⁸ is (C₁-C₆)oxaalkyl. In still another further example, R¹⁸ is oxo(C₁-C₆)azaalkene. In an example, R¹⁸ is (C₃-C₁₀)carbocycle. In another example, R¹⁸ is heterocycle is optionally substituted with one or more of —N(R¹⁵)₂. In yet another example, R¹⁸ is B(OH)₂.

In an example, R¹⁹ is hydrogen. In another example, R¹⁹ is halogen. In yet another example, R¹⁹ is CN. In still another example, R¹⁹ is C(═O)N(R¹⁵)₂, In a further example, R¹⁹ is C(═O)OR¹⁵. In yet a further example, R¹⁹ is C(═O)R¹⁵. In still a further example, R¹⁹ is —N(R¹⁵)₂. In still a further example, R¹⁹ is (C₁-C₆)alkoxy. In another further example, R¹⁹ is (C₁-C₆)thiaalkyl. In yet another further example, R¹⁹ is (C₁-C₆)oxaalkyl. In still another further example, R¹⁹ is oxo(C₁-C₆)azaalkene. In an example, R¹⁹ is (C₃-C₁₀)carbocycle. In another example, R¹⁹ is heterocycle is optionally substituted with one or more of —N(R¹⁵)₂. In yet another example, R¹⁹ is B(OH)₂.

In an example, R²⁰ is hydrogen. In another example, R²⁰ is halogen. In yet another example, R²⁰ is CN. In still another example, R²⁰ is C(═O)N(R¹⁵)₂, In a further example, R²⁰ is C(═O)OR¹⁵. In yet a further example, R²⁰ is C(═O)R¹⁵. In still a further example, R²⁰ is —N(R¹⁵)₂. In still a further example, R²⁰ is (C₁-C₆)alkoxy. In another further example, R²⁰ is (C₁-C₆)thiaalkyl. In yet another further example, R²⁰ is (C₁-C₆)oxaalkyl. In still another further example, R²⁰ is oxo(C₁-C₆)azaalkene. In an example, R²⁰ is (C₃-C₁₀)carbocycle. In another example, R²⁰ is heterocycle is optionally substituted with one or more of —N(R¹⁵)₂. In yet another example, R²⁰ is B(OH)₂.

In an example, R¹² is heterocycle optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂; and R¹⁴ is heterocycle optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂.

In an example, R¹⁴ is phenyl.

In an example, the compound of formula II, or a pharmaceutically acceptable salt thereof, may have the following structure:

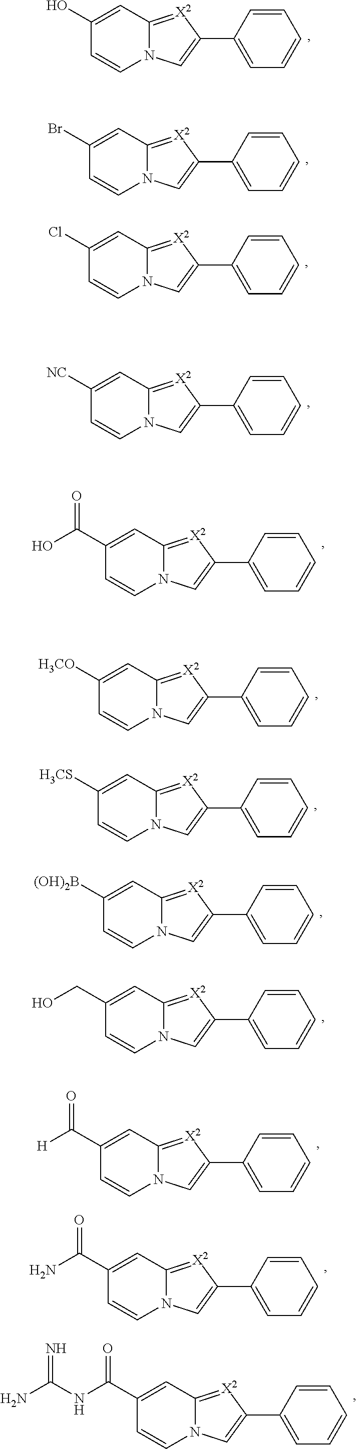

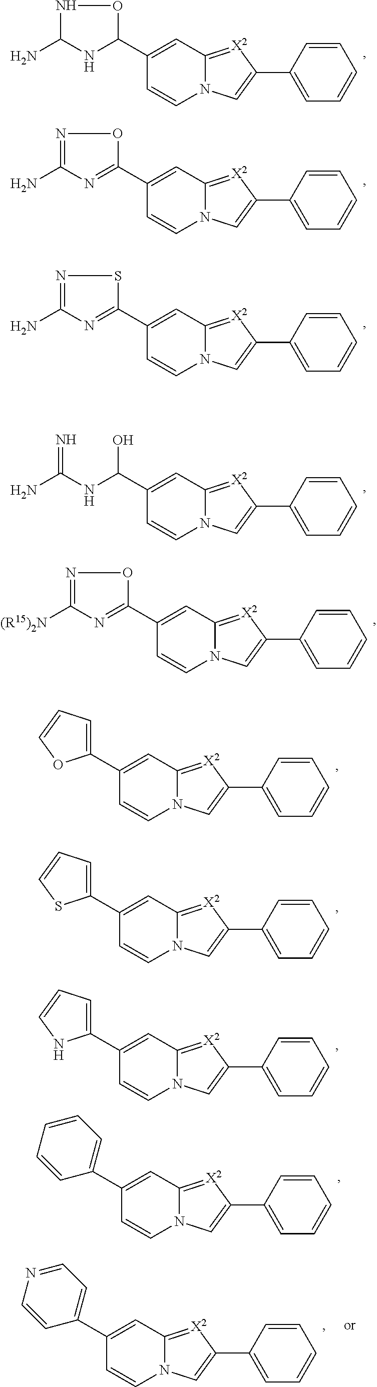

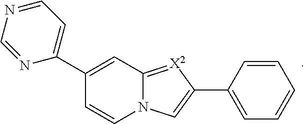

In an example, the compound of formula II, or a pharmaceutically acceptable salt thereof, may have the following structure:

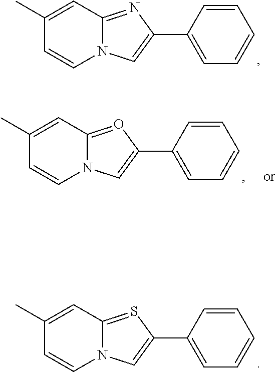

In an aspect, disclosed is a pharmaceutical composition including a compound of formula I, as described herein, and a pharmaceutically acceptable excipient.

In another aspect, disclosed is a pharmaceutical composition including a compound of formula II, as described herein, and a pharmaceutically acceptable excipient.

In yet another aspect, provided is a method of treatment, including administering a compound of formula I, as described herein, to a subject wherein the subject suffers from kidney disease or is at risk of kidney disease.

In still another aspect, provided is a method of treatment, including administering a compound of formula II, as described herein, to a subject wherein the subject suffers from kidney disease or is at risk of kidney disease.

In a further aspect, provided is a method of treatment, including administering a pharmaceutical composition, as described herein, to a subject wherein the subject suffers from kidney disease or is at risk of kidney disease.

### Abbreviations and Definitions

Unless defined otherwise, all technical and scientific terms used herein have the same meaning as is commonly understood by one of ordinary skill in the art to which this disclosure belongs. A comprehensive list of abbreviations utilized by organic chemists (i.e., persons of ordinary skill in the art) appears in the first issue of each volume of the Journal of Organic Chemistry. The list, which is typically presented in a table entitled “Standard List of Abbreviations” is incorporated herein by reference. In the event that there is a plurality of definitions for terms cited herein, those in this section prevail unless otherwise stated.

As used herein, the terms “comprising” and “including,” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

Throughout this specification the terms and substituents retain their definitions.

Unless otherwise specified herein, “hydrocarbon” or hydrocarbyl (as a substituent), means any substituent comprised of hydrogen and carbon as the only elemental constituents. If not otherwise limited, (C₁-Cₙ) hydrocarbon, wherein n may be any integer from 1 to 20 or higher, is intended to include alkyl, cycloalkyl, polycycloalkyl, alkenyl, alkynyl, aryl, and combinations thereof. Non-limiting examples of a hydrocarbon include cyclopropylmethyl, benzyl, phenethyl, cyclohexylmethyl, adamantyl, camphoryl, and naphthylethyl. Hydrocarbyl refers to any substituent comprised of hydrogen and carbon as the only elemental constituents. Aliphatic hydrocarbons are hydrocarbons that are not aromatic; they may be saturated or unsaturated, cyclic, linear, or branched. Examples of aliphatic hydrocarbons include isopropyl, 2-butenyl, 2-butynyl, cyclopentyl, norbornyl, etc. Aromatic hydrocarbons include benzene (phenyl), naphthalene (naphthyl), anthracene, etc.

Unless otherwise specified herein, “alkyl” (or alkylene) is intended to include linear or branched saturated hydrocarbon structures and combinations thereof. Non-limiting examples of alkyl include methyl, ethyl, propyl, isopropyl, n-butyl, s-butyl, t-butyl, and the like. (C₁-C₁₀)alkyl includes all combinations therein, i.e., (C₁-C₂)alkyl, (C₁-C₃)alkyl, (C₁-C₄)alkyl, (C₁-C5)alkyl, (C₁-C₆)alkyl, (C₁-C₇)alkyl, (C₁-C₈)alkyl, (C₁-C₉)alkyl, (C₂-C₃)alkyl, (C₂-C₄)alkyl, (C₂-C5)alkyl, (C₂-C₆)alkyl, (C₂-C₇)alkyl, (C₂-C₈)alkyl, (C₂-C₉)alkyl, (C₂-C₁₀)alkyl, (C₃-C₄)alkyl, (C₃-C5)alkyl, (C₃-C₆)alkyl, (C₃-C₇)alkyl, (C₃-C₈)alkyl, (C₃-C₉)alkyl, (C₃-C₁₀)alkyl, (C₄-C₅)alkyl, (C₄-C₆)alkyl, (C₄-C₇)alkyl, (C₄-C₈)alkyl, (C₄-C₉)alkyl, (C₄-C₁₀)alkyl, (C₅-C₆)alkyl, (C₅-C₇)alkyl, (C₅-C8)alkyl, (C₅-C₉)alkyl, (C₅-C₁₀)alkyl, (C₆-C₇)alkyl, (C₆-C₈)alkyl, (C₆-C₉)alkyl, (C₆-C₁₀)alkyl, (C₇-C₈)alkyl, (C₇-C₉)alkyl, (C₇-C₁₀)alkyl, (C₈-C₉)alkyl, (C₈-C₁₀)alkyl, (C₉-C₁₀)alkyl, (C₁)alkyl, (C₂)alkyl, (C₃)alkyl, (C₄)alkyl, (C₅)alkyl, (C₆)alkyl, (C₇)alkyl, (Ca)alkyl, (C₉)alkyl, and (C₁₀)alkyl.

Unless otherwise specified herein, “oxaalkyl” is intended to include alkyl residues in which one or more carbons (and their associated hydrogens) have been replaced by oxygen. Non-limiting examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127 (a)], i.e., it refers to compounds in which the oxygen is bonded via a single bond to its adjacent atoms (forming ether bonds); it does not refer to doubly bonded oxygen, as would be found in carbonyl groups. (C₁-C₁₀)oxaalkyl includes all combinations therein, i.e., (C₁-C₂)oxaalkyl, (C₁-C₃)oxaalkyl, (C₁-C₄)oxaalkyl, (C₁-C₅)oxaalkyl, (C₁-C₆)oxaalkyl, (C₁-C₇)oxaalkyl, (C₁-C₈)oxaalkyl, (C₁-C₉)oxaalkyl, (C₂-C₃)oxaalkyl, (C₂-C₄)oxaalkyl, (C₂-C₅)oxaalkyl, (C₂-C₆)oxaalkyl, (C₂-C₇)oxaalkyl, (C₂-C₈)oxaalkyl, (C₂-C₉)oxaalkyl, (C₂-C₁₀)oxaalkyl, (C₃-C₄)oxaalkyl, (C₃-C₅)oxaalkyl, (C₃-C₆)oxaalkyl, (C₃-C₇)oxaalkyl, (C₃-C₈)oxaalkyl, (C₃-C₉)oxaalkyl, (C₃-C₁₀)oxaalkyl, (C₄-C₅)oxaalkyl, (C₄-C₆)oxaalkyl, (C₄-C₇)oxaalkyl, (C₄-C₈)oxaalkyl, (C₄-C₉)oxaalkyl, (C₄-C₁₀)oxaalkyl, (C₅-C₆)oxaalkyl, (C₅-C₇)oxaalkyl, (C₅-C₈)oxaalkyl, (C₅-C₉)oxaalkyl, (C₅-C₁₀)oxaalkyl, (C₆-C₇)oxaalkyl, (C₆-C₅)oxaalkyl, (C₆-C₉)oxaalkyl, (C₆-C₁₀)oxaalkyl, (C₇-C₈)oxaalkyl, (C₇-C₉)oxaalkyl, (C₇-C₁₀)oxaalkyl, (C₈-C₉)oxaalkyl, (C₈-C₁₀)oxaalkyl, (C₉-C₁₀)oxaalkyl, (C₁)oxaalkyl, (C₂)oxaalkyl, (C₃)oxaalkyl, (C₄)oxaalkyl, (C₅)oxaalkyl, (C₆)oxaalkyl, (C₇)oxaalkyl, (C₅)oxaalkyl, (C₉)oxaalkyl, and (C₁₀)oxaalkyl.

Unless otherwise specified, azaalkyl is intended to include alkyl residues in which one or more carbons (and their associated hydrogens) have been replaced by nitrogen. Non-limiting examples include ethylaminoethyl. (C₁-C₁₀)azaalkyl includes all combinations therein, i.e., (C₁-C₂)azaalkyl, (C₁-C₃)azaalkyl, (C₁-C₄)azaalkyl, (C₁-C₅)azaalkyl, (C₁-C₆)azaalkyl, (C₁-C₇)azaalkyl, (C₁-C₈)azaalkyl, (C₁-C₉)azaalkyl, (C₂-C₃)azaalkyl, (C₂-C₄)azaalkyl, (C₂-C₅)azaalkyl, (C₂-C₆)azaalkyl, (C₂-C₇)azaalkyl, (C₂-C₈)azaalkyl, (C₂-C₉)azaalkyl, (C₂-C₁₀)azaalkyl, (C₃-C₄)azaalkyl, (C₃-C₅)azaalkyl, (C₃-C₆)azaalkyl, (C₃-C₇)azaalkyl, (C₃-C₈)azaalkyl, (C₃-C₉)azaalkyl, (C₃-C₁₀)azaalkyl, (C₄-C₅)azaalkyl, (C₄-C₆)azaalkyl, (C₄-C₇)azaalkyl, (C₄-C₈)azaalkyl, (C₄-C₉)azaalkyl, (C₄-C₁₀)azaalkyl, (C₅-C₆)azaalkyl, (C₅-C₇)azaalkyl, (C₅-C₈)azaalkyl, (C₅-C₉)azaalkyl, (C₅-C₁₀)azaalkyl, (C₆-C₇)azaalkyl, (C₆-C₈)azaalkyl, (C₆-C₉)azaalkyl, (C₆-C₁₀)azaalkyl, (C₇-C₈)azaalkyl, (C₇-C₉)azaalkyl, (C₇-C₁₀)azaalkyl, (C₈-C₉)azaalkyl, (C₈-C₁₀)azaalkyl, (C₉-C₁₀)azaalkyl, (C₁)azaalkyl, (C₇)azaalkyl, (C₃)azaalkyl, (C₄)azaalkyl, (C₅)azaalkyl, (C₆)azaalkyl, (C₇)azaalkyl, (Ca)azaalkyl, (C)azaalkyl, and (C₁₀)azaalkyl.

Unless otherwise specified, thiaalkyl is intended to include alkyl residues in which one or more carbons (and their associated hydrogens) have been replaced by sulfur. Non-limiting examples include methylthiopropyl. (C₁-C₁₀) thiaalkyl includes all combinations therein, i.e., (C₁-C₂) thiaalkyl, (C₁-C₃)thiaalkyl, (C₁-C₄)thiaalkyl, (C₁-C₅)thiaalkyl, (C₁-C₆)thiaalkyl, (C₁-C₇)thiaalkyl, (C₁-C₈)thiaalkyl, (C₁-C₉)thiaalkyl, (C₂-C₃)thiaalkyl, (C₂-C₄)thiaalkyl, (C₂-C₅)thiaalkyl, (C₂-C₆)thiaalkyl, (C₂-C₇)thiaalkyl, (C₂-C₈)thiaalkyl, (C₂-C₉)thiaalkyl, (C₂-C₁₀)thiaalkyl, (C₃-C₄)thiaalkyl, (C₃-C₅)thiaalkyl, (C₃-C₆)thiaalkyl, (C₃-C₇)thiaalkyl, (C₃-C₈)thiaalkyl, (C₃-C₉)thiaalkyl, (C₃-C₁₀)thiaalkyl, (C₄-C₅)thiaalkyl, (C₄-C₆)thiaalkyl, (C₄-C₇)thiaalkyl, (C₄-C₈)thiaalkyl, (C₄-C₉)thiaalkyl, (C₄-C₁₀)thiaalkyl, (C₅-C₆)thiaalkyl, (C₅-C₇)thiaalkyl, (C₅-C₈)thiaalkyl, (C₅-C₉)thiaalkyl, (C₅-C₁₀)thiaalkyl, (C₆-C₇)thiaalkyl, (C₆-C₈)thiaalkyl, (C₆-C₉)thiaalkyl, (C₆-C₁₀)thiaalkyl, (C₇-C₈)thiaalkyl, (C₇-C₉)thiaalkyl, (C₇-C₁₀)thiaalkyl, (C₈-C₉)thiaalkyl, (C₈-C₁₀)thiaalkyl, (C₉-C₁₀)thiaalkyl, (C₁)thiaalkyl, (C₂)thiaalkyl, (C₃)thiaalkyl, (C₄)thiaalkyl, (C₅)thiaalkyl, (C₆)thiaalkyl, (C₇)thiaalkyl, (C₈)thiaalkyl, (C₉)thiaalkyl, and (C₁₀)thiaalkyl.

Unless otherwise specified herein, “carbocycle” is intended to include ring systems in which the ring atoms are all carbon but of any oxidation state. If not otherwise limited, “carbocycle” is intended to include both non-aromatic and aromatic systems. In addition, unless otherwise specified herein, “carbocycle” is intended to include monocycles, bicycles, and polycycles. In a non-limiting example, (C₃-C₁₀)carbocycle may refer to cyclopropane, cyclohexane, benzene, phenyl, cyclopentadiene, cyclohexene, norbornane, decalin, naphthalene, indane, and the like.

Unless otherwise specified herein, “cycloalkyl” is a subset of hydrocarbyl and is intended to include cyclic hydrocarbon structures. If not otherwise limited, “cycloalkyl” may include cyclic alkyl groups of from 3 to 8 carbon atoms or from 3 to 6 carbon atoms. Non-limiting examples of cycloalkyl include cy-propyl, cy-butyl, cy-pentyl, norbornyl, and the like. (C₃-C₁₀)cycloalkyl includes all combinations therein, i.e., (C₃-C₄)cycloalkyl, (C₃-C₅)cycloalkyl, (C₃-C₆)cycloalkyl, (C₃-C₇)cycloalkyl, (C₃-C₈)cycloalkyl, (C₃-C₉)cycloalkyl, (C₃-C₁₀)cycloalkyl, (C₄-C₅)cycloalkyl, (C₄-C₆)cycloalkyl, (C₄-C₇)cycloalkyl, (C₄-C₈)cycloalkyl, (C₄-C₉)cycloalkyl, (C₄-C₁₀)cycloalkyl, (C₅-C₆)cycloalkyl, (C₅-C₇)cycloalkyl, (C₅-C₈)cycloalkyl, (C₅-C₉)cycloalkyl, (C₅-C₁₀)cycloalkyl, (C₆-C₇)cycloalkyl, (C₆-C₅)cycloalkyl, (C₆-C₉)cycloalkyl, (C₆-C₁₀)cycloalkyl, (C₇-C₈)cycloalkyl, (C₇-C₉)cycloalkyl, (C₇-C₁₀)cycloalkyl, (C₈-C₉)cycloalkyl, (C₈-C₁₀)cycloalkyl, (C₉-C₁₀)cycloalkyl, (C₃)cycloalkyl, (C₄)cycloalkyl, (C₅)cycloalkyl, (C₆)cycloalkyl, (C₇)cycloalkyl, (Ca)cycloalkyl, (C₉)cycloalkyl, and (C₁₀)cycloalkyl.

Heteroaryl is a subset of heterocycle in which the heterocycle is aromatic. In some instances, the heteroaryl contains 4, 5, 6, or 7 ring members. In some instances, the heteroaryl is bicyclic and contains 8, 9, 10, or 11 total ring members. Non-limiting examples include furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, benzothiazole, triazole, tetrazole, pyridine, quinoline, isoquinoline, pyrazine, quinoxaline, acridine, pyrimidine, quinazoline, pyridazine, cinnoline, phthalazine, and triazine. Non-limiting examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

Unless otherwise specified herein, “heterocycle” means an aliphatic or aromatic carbocycle residue in which from one to four carbons is replaced by a heteroatom selected from the group consisting of N, O, and S. The nitrogen and sulfur heteroatoms may optionally be oxidized, and the nitrogen heteroatom may optionally be quaternized. If not otherwise limited, “heterocycle” may be non-aromatic (e.g., heteroaliphatic) or aromatic (heteroaryl). Unless otherwise specified herein, “heterocycle” refers to monocycles, bicycles, spirocycles, and polycycles. Non-limiting examples of heterocycles include pyridine, pyrimidine, pyrrole, thiophene, furan, oxadiazol, thiadiazole, pyrrolidine, pyrazole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridazine, oxazole, oxazoline, isoxazole, dioxane, tetrahydrofuran and the like. Examples of heterocyclyl residues include piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl (also historically called thiophenyl), benzothienyl, thiamorpholinyl, oxadiazolyl, triazolyl and tetrahydroquinolinyl.

Unless otherwise specified herein, “alkoxy” or “alkoxyl” refers to groups of from 1 to 20 carbon atoms, from 1 to 10 carbon atoms, or from 1 to 6 carbon atoms of a straight, branched, or cyclic configuration, and combinations thereof, attached to the parent structure through an oxygen. Non-limiting examples include methoxy, ethoxy, propoxy, isopropoxy cyclopropyloxy, cyclohexyloxy, methylenedioxy, ethylenedioxy, and the like. (C₁-C₁₀)alkoxy includes all combinations therein, i.e., (C₁-C₂)alkoxy, (C₁-C₃)alkoxy, (C₁-C₄)alkoxy, (C₁-C₅)alkoxy, (C₁-C₆)alkoxy, (C₁-C₇)alkoxy, (C₁-C₈)alkoxy, (C₁-C₉)alkoxy, (C₂-C₃)alkoxy, (C₂-C₄)alkoxy, (C₂-C₅)alkoxy, (C₂-C₆)alkoxy, (C₂-C₇)alkoxy, (C₂-C₈)alkoxy, (C₂-C₉)alkoxy, (C₂-C₁₀)alkoxy, (C₃-C₄)alkoxy, (C₃-C₅)alkoxy, (C₃-C₆)alkoxy, (C₃-C₇)alkoxy, (C₃-C₈)alkoxy, (C₃-C₉)alkoxy, (C₃-C₁₀)alkoxy, (C₄-C₅)alkoxy, (C₄-C₆)alkoxy, (C₄-C₇)alkoxy, (C₄-C₈)alkoxy, (C₄-C₉)alkoxy, (C₄-C₁₀)alkoxy, (C₅-C₆)alkoxy, (C₅-C₇)alkoxy, (C₅-C₈)alkoxy, (C₅-C₉)alkoxy, (C₅-C₁₀)alkoxy, (C₆-C₇)alkoxy, (C₆-C₅)alkoxy, (C₆-C₉)alkoxy, (C₆-C₁₀)alkoxy, (C₇-C₈)alkoxy, (C₇-C₉)alkoxy, (C₇-C₁₀)alkoxy, (C₈-C₉)alkoxy, (C₈-C₁₀)alkoxy, (C₉-C₁₀)alkoxy, (C₁)alkoxy, (C₂)alkoxy, (C₃)alkoxy, (C+)alkoxy, (C₅)alkoxy, (C₆)alkoxy, (C₇)alkoxy, (C₅)alkoxy, (C₉)alkoxy, and (C₁₀)alkoxy.

Unless otherwise specified, acyl refers to formyl and to groups of 1, 2, 3, 4, 5, 6, 7 or 8 carbon atoms of a straight, branched, or cyclic configuration, saturated or unsaturated, and aromatic, and combinations thereof, attached to the parent structure through a carbonyl functionality. One or more carbons in the acyl residue may be replaced by nitrogen, oxygen or sulfur as long as the point of attachment to the parent remains at the carbonyl. Non-limiting examples include acetyl, benzoyl, propionyl, isobutyryl, t-butoxycarbonyl, benzyloxycarbonyl, and the like. The double bonded oxygen, when referred to as a substituent itself is called “oxo”.

Arylalkyl refers to a substituent in which an aryl residue is attached to the parent structure through alkyl. Non-limiting examples include benzyl, phenethyl and the like. Heteroarylalkyl refers to a substituent in which a heteroaryl residue is attached to the parent structure through alkyl. In one embodiment, the alkyl group of an arylalkyl or a heteroarylalkyl is an alkyl group of from 1 to 6 carbons. Non-limiting examples include pyridinylmethyl, pyrimidinylethyl, and the like.

An oxygen heterocycle is a heterocycle containing at least one oxygen in the ring; it may contain additional oxygens, as well as other heteroatoms. A sulphur heterocycle is a heterocycle containing at least one sulphur in the ring; it may contain additional sulphurs, as well as other heteroatoms. Oxygen heteroaryl is a subset of oxygen heterocycle; non-limiting examples include furan and oxazole. Sulphur heteroaryl is a subset of sulphur heterocycle; non-limiting examples include thiophene and thiazine. A nitrogen heterocycle is a heterocycle containing at least one nitrogen in the ring; it may contain additional nitrogens, as well as other heteroatoms. Non-limiting examples include piperidine, piperazine, morpholine, pyrrolidine and thiomorpholine. Nitrogen heteroaryl is a subset of nitrogen heterocycle; non-limiting examples include pyridine, pyrrole and thiazole.

The term “halogen” means fluorine, chlorine, bromine, or iodine atoms. In an example, halogen may be a chlorine atom. In another example, halogen may be a bromine atom. In yet another example, halogen may be an iodine atom. In still another example, halogen may be a fluorine atom.

The terms “haloalkyl” and “haloalkoxy” mean alkyl or alkoxy, respectively, substituted with one or more halogen atoms. The terms “alkylcarbonyl” and “alkoxycarbonyl” mean —C(═O)alkyl or —C(O)alkoxy, respectively.

Unless otherwise specified herein, the term “optionally substituted” may be used interchangeably with “unsubstituted or substituted.” The term “substituted” may refer to the replacement of one or more hydrogen atoms in a specified group with a specified radical. In a non-limiting example, “optionally substituted heterocyclyl” may refer to an unsubstituted or substituted heterocyclyl, and “substituted heterocyclyl” may refer to heterocyclyl wherein one or more H atoms in each residue are replaced with

B(OH)₂, halogen, haloalkyl, alkyl, acyl, alkoxyalkyl, hydroxyalkyl, carbonyl, phenyl, heteroaryl, benzenesulfonyl, (C₃-C₁₀) carbocyclyl, hydroxy, alkoxy, oxaalkyl, thiaalkyl, haloalkoxy, oxaalkyl, carboxy, alkoxycarbonyl [—C(═O)O-alkyl], aminocarbonyl (also known as carboxamido) [—C(═O)NH2], alkoxycarbonylamino [HNC(═O)O-alkyl], alkylaminocarbonyl [—C(═O)NH-alkyl], dialkylaminocarbonyl, cyano, acetoxy, nitro, amino, alkylamino, dialkylamino, dialkylaminoalkyl, dialkylaminoalkoxy, heterocyclylalkoxy, arylalkyl, (cycloalkyl)alkyl, heterocyclyl, heterocyclylalkyl, alkylaminoalkyl (including cycloalkylaminoalkyl), heterocyclylaminoalkyl, heterocyclylalkylaminoalkyl, cycloalkylaminoalkyl, cycloalkylalkylaminoalkyl, arylaminoalkyl, arylalkylaminoalkyl, mercapto, alkylthio, sulfoxide, sulfone, sulfonylamino, alkylsulfinyl, alkylsulfonyl, acylaminoalkyl, acylaminoalkoxy, acylamino, aryl, benzyl, heterocyclyl, phenoxy, benzyloxy, heteroaryloxy, hydroxyimino, alkoxyimino, aminosulfonyl, trityl, amidino, guanidino, ureido, benzyloxyphenyl, (C₁₋₁₀)hydrocarbyl, —SO2alkyl, —SO2NH2, oxo(C₁-C₆)azaalkene, or —SO2NHalkyl. “Oxo” may also be included among the substituents referred to in “optionally substituted”; it will be appreciated by persons of skill in the art that, because oxo is a divalent radical, there are circumstances in which it will not be appropriate as a substituent (e.g., on phenyl). In an embodiment, 1, 2, or 3 hydrogen atoms may be replaced with a specified radical. In the case of alkyl and cycloalkyl, more than three hydrogen atoms may be replaced by fluorine; indeed, all available hydrogen atoms may be replaced by fluorine.

It is understood that any alkyl, alkenyl, alkynyl, cycloalkyl and cycloalkenyl moiety described herein can also be an aliphatic group, an alicyclic group or a heterocyclic group. An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may be used in place of the alkyl, alkenyl, alkynyl, alkylene, alkenylene, and alkynylene groups described herein.

Substituents Rⁿ are generally defined when introduced and retain that definition throughout the specification and in all independent claims.

Unless otherwise stated or depicted, structures depicted herein are also meant to include all stereoisomeric (e.g., enantiomeric, diastereomeric, and cis-trans isomeric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and cis-trans isomeric (or conformational) mixtures of the present compounds are within the scope of this disclosure. Unless otherwise stated, all tautomeric forms of the compounds disclosed herein are within the scope of this disclosure.

Additionally, unless otherwise stated, structures depicted herein are also meant to include compounds that differ only in the presence of one or more isotopically enriched atoms. Radioisotopes of hydrogen, carbon, phosphorous, fluorine, and chlorine include ²H, ³H, ¹³C, ¹⁴C, ¹⁵N, ³⁵S, ¹⁸F, and ³⁶Cl, respectively. Compounds that contain those radioisotopes and/or other radioisotopes of other atoms are within the scope of this disclosure. Such compounds are useful, for example, as analytical tools or probes in biological assays. Radiolabeled compounds of the present disclosure can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

Preparation of compounds can involve the protection and deprotection of various chemical groups. The need for protection and deprotection, and the selection of appropriate protecting groups, can be readily determined by one skilled in the art. Suitable groups for that purpose are discussed in standard textbooks in the field of chemistry, such as Protective Groups in Organic Synthesis by T. W. Greene and P. G. M. Wuts [John Wiley & Sons, New York, 1999], in Protecting Group Chemistry, 1^{st} Ed., Oxford University Press, 2000; and in March's Advanced Organic chemistry: Reactions, Mechanisms, and Structure, 5^{th} Ed., Wiley-Interscience Publication, 2001.

As used herein, and as would be understood by the person of skill in the art, the recitation of “a compound”—unless expressly further limited—is intended to include salts of that compound. In a particular embodiment, the term “compound of formula” refers to the compound or a pharmaceutically acceptable salt thereof.

The terms “subject” or “subject in need thereof” are used interchangeably herein. These terms refer to a patient who has been diagnosed with the underlying disorder to be treated. The subject may currently be experiencing symptoms associated with the disorder or may have experienced symptoms in the past. Additionally, a “subject in need thereof” may be a patient at risk of developing a particular disease, or to a patient reporting one or more of the physiological systems of a disease, even though a diagnosis of this disease may not have been made. A subject may be a patient diagnosed with a kidney disease or at risk of contracting a kidney disease, such as a subject with a genetic or other predisposition for developing a kidney disease. In an example, the kidney disease may be a glomerular disease. In another example, the kidney disease may be glomerulonephritis. In yet another example, the kidney disease may be glomerulosclerosis. In still another example, the kidney disease may be focal segmental glomerulosclerosis (FSGS). In a further example, the kidney disease may be kidney fibrosis. In yet a further example, the kidney disease may be cystinosis. In still a further example, the kidney disease may be an autoimmune disease.

As used herein, the terms “treatment” or “treating” are used interchangeably. These terms refer to an approach for obtaining beneficial or desired results including, but not limited to, therapeutic benefit. Therapeutic benefit includes eradication or amelioration of the underlying disorder being treated; it also includes the eradication or amelioration of one or more of the symptoms associated with the underlying disorder such that an improvement is observed in the patient, notwithstanding that the patient may still be afflicted with the underlying disorder. In an example, treatment may include administering a compound, pharmaceutically acceptable salt thereof, or pharmaceutical composition comprising one or more of the foregoing, to a subject in need thereof. For example, the subject may be diagnosed with a kidney disease, or at risk of developing a kidney disease. In an example, the kidney disease may be a glomerular disease. In another example, the kidney disease may be glomerulonephritis. In yet another example, the kidney disease may be glomerulosclerosis. In still another example the kidney disease may be focal segmental glomerulosclerosis (FSGS). In a further example, the kidney disease may be kidney fibrosis. In yet a further example, the kidney disease may be cystinosis. In still a further example, the kidney disease may be an autoimmune disease.

The term “pharmaceutically acceptable salt” refers to salts prepared from pharmaceutically acceptable non-toxic acids or bases including inorganic acids and bases and organic acids and bases. When the compounds of the present disclosure are basic, salts may be prepared from pharmaceutically acceptable non-toxic acids including inorganic and organic acids. Suitable pharmaceutically acceptable acid addition salts for the compounds of the present disclosure include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present disclosure include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

Also provided herein is a pharmaceutical composition comprising a compound disclosed herein, or a pharmaceutically acceptable salt thereof, together with one or more pharmaceutically acceptable excipients thereof. The excipient(s) must be “acceptable” in the sense of being compatible with any other ingredients of the formulation and not deleterious to the recipient thereof.

A pharmaceutical composition including a compound of Formula I or II includes, as a non-limiting example, such compound in a lyophilized or dry form such that dissolving such dry form in solvent, including upon oral administration to a subject, such compound would bind with copper as administered therewith in solution. Formulations for administration to a subject include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of a recipient or intended purpose of the administration. A formulation may conveniently be presented in unit dosage form and may be prepared by any of the methods well known in the art of pharmacy. A method may include a step of bringing into association a compound of Formula I or II or a pharmaceutically acceptable salt thereof (“active ingredient”) with a carrier which constitutes one or more accessory ingredients. In general, formulations may be prepared by uniformly and intimately bringing into association an active ingredient with liquid carriers or finely divided solid carriers or both and then, if necessary, shaping the product into the desired formulation.

Formulations of the present disclosure suitable for oral administration may be presented as discrete units such as capsules, cachets or tablets each containing a predetermined amount of an active ingredient; as a powder or granules; as a solution or a suspension in an aqueous liquid or a non-aqueous liquid; or as an oil-in-water liquid emulsion or a water-in-oil liquid emulsion. A compound of Formula I or II may also be presented as a bolus, electuary or paste. For oral or other administration, a compound of Formula I or II may be suspended in a solution, or dissolved in a solvent, such as alcohol, DMSO, water, saline, or other solvent, which may be further diluted or dissolved in another solution or solvent, and may or may contain a carrier or other excipient in some examples.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral or other administration include aqueous and non-aqueous sterile injection solutions which may contain anti-oxidants, buffers, bacteriostats and solutes which render a formulation isotonic with the blood of the intended recipient. Formulations for parenteral or other administration also may include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

For the purpose of the present disclosure, a “pure” or “substantially pure” enantiomer is intended to mean that the enantiomer is at least 95% of the configuration shown and 5% or less of other enantiomers. Similarly, a “pure” or “substantially pure” diastereomer is intended to mean that the diastereomer is at least 95% of the relative configuration shown and 5% or less of other diastereomers.

The pharmaceutical compositions disclosed herein may include one or more pharmaceutically acceptable excipients, including, but not limited to, one or more binders, bulking agents, buffers, stabilizing agents, surfactants, wetting agents, lubricating agents, diluents, disintegrants, viscosity enhancing or reducing agents, emulsifiers, suspending agents, preservatives, antioxidants, opacifying agents, glidants, processing aids, colorants, sweeteners, taste-masking agents, perfuming agents, flavoring agents, diluents, polishing agents, polymer matrix systems, plasticizers and other known additives to provide an elegant presentation of the drug or aid in the manufacturing of a medicament or pharmaceutical product comprising a composition of the present disclosure. Non-limiting examples of carriers and excipients well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

As used herein, the term “effective amount” means an amount of a compound of Formula I pharmaceutical agent that may elicit a biological or medical response of a cell, tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula I, as well as salts, solvates, and physiological functional derivatives thereof, may be administered as the raw chemical. Additionally, the active ingredient may be presented as a pharmaceutical composition.

Pharmaceutical compositions as disclosed herein may include an effective amount of a compound of Formula I or II and optionally one or more additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains a compound of Formula I or II and optionally one or more additional active ingredient will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

The compounds disclosed herein are synthesized as follows:

### General Synthetic Schemes

The compounds disclosed herein were prepared by methods well known in the art of synthetic organic chemistry. Preparation of compounds may involve the protection and deprotection of various sensitive or reactive chemical groups. The need for protection and deprotection, and the selection of appropriate protecting groups, can be readily determined by one skilled in the art. Suitable groups for that purpose are discussed in standard textbooks in the field of chemistry, such as Protective Groups in Organic Synthesis by T. W. Greene and P. G. M. Wuts [John Wiley & Sons, New York, 1999], in Protecting Group Chemistry, 1^{st} Ed., Oxford University Press, 2000; and in March's Advanced Organic chemistry: Reactions, Mechanisms, and Structure, 5^{th} Ed., Wiley-Interscience Publication, 2001. The protecting groups may be removed at a convenient subsequent stage using methods well known in the art.

In general, compounds can be prepared by the methods illustrated in the general reaction schemes described below, or by modifications thereof, using readily available starting materials, reagents, and conventional synthetic procedures. However, those skilled in the art will recognize that other methods may also be suitable. Also, in these reactions, it is possible to make use of variants that are in themselves known but are not mentioned here.

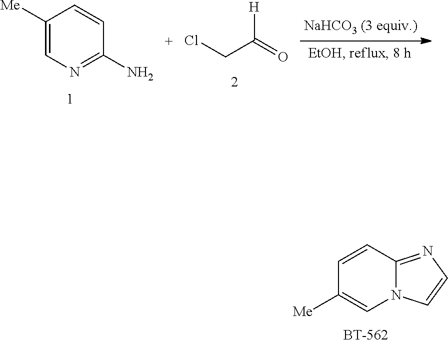

### Procedure for the Synthesis of 6-methylimidazo[1,2-α]pyridine (BT-562)

A clean oven dried 20 mL round bottom flask was charged with (1) (1 equiv.), (2) (3 equiv.), and NaHCO₃ (3 equiv.), in ethanol (2 mL). The reaction mixture was refluxed for 8 h. Reaction progress was monitored by TLC. After completion of the reaction, the reaction mass was allowed to cool at ambient temperature, then ethanol was evaporated, diluted with water (10 mL) and extracted with EtOAc (3×10 mL). The combined organic layer was dried with anhydrous Na₂SO₄ and evaporated under reduced pressure. The crude material was purified by column chromatography (100% ethyl acetate). ¹H NMR (400 MHZ, DMSO) δ 8.37 (s, 1H), 7.89 (s, 1H), 7.57 (d, J=0.8 Hz, 1H), 7.50 (d, J=9.2 Hz, 1H), 7.14 (dd, J=9.2, 1.3 Hz, 1H), 2.27 (s, 3H). ¹³C NMR (100 MHZ, DMSO) δ 143.47, 132.27, 128.65, 124.99, 122.09, 116.27, 113.35, 17.93.

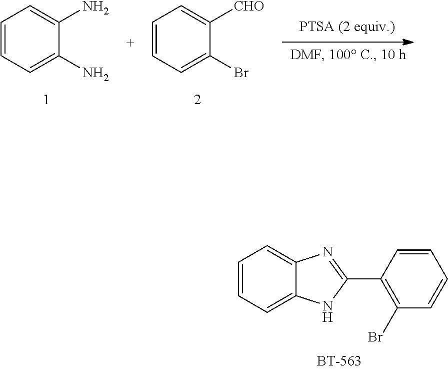 [SMILES: Nc1ccccc1N ; O=Cc1ccccc1Br ; Brc1ccccc1-c1nc2ccccc2[nH]1]

### Procedure for the Synthesis of 2-(2-bromophenyl)-1H-benzo[d]imidazole (BT-563)

A clean oven dried 25 mL round bottom flask was charged with 1 (1 equiv.), 2 (1.1 equiv.), p-toluenesulfonic acid (PTSA) (2 equiv.) and DMF (3 mL). The resulted reaction mixture was stirred at 100° C. for 10 h. Reaction progress was monitored by TLC. After completion, the reaction mass was allowed to cool at ambient temperature, diluted with water and extracted with EtOAc (2×10 mL). The combined organic layer was dried with anhydrous Na₂SO₄ and evaporated under reduced pressure. The crude material was purified by column chromatography on silica gel (100-200 mesh). Off-White solid (Yield =75%), ¹H NMR (400MHz, DMSO) δ 12.79 (s, 1H), 8.24 (d, J=7.2 Hz, 1H), 7.88 (d, J=7.8 Hz, 1H), 7.82 (dd, J=7.6, 1.2 Hz, 1H), 7.64-7.50 (m, 5H).13C NMR (100 MHz, DMSO) δ 150.9, 133.9, 132.9, 132.7, 131.8, 130.6, 130.3, 129.4, 128.3, 126.9, 122.0. HRMS (ESI) calcd for C₁₃H₁₀BrN₂ 273.0022found 273.0105 [M+H]⁺.

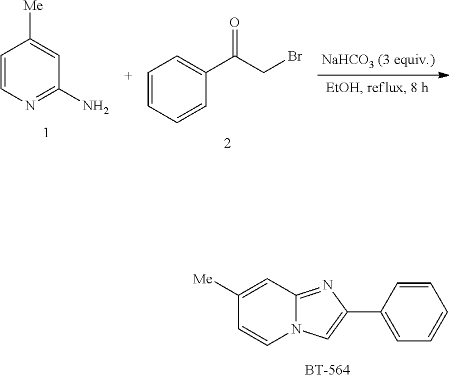 [SMILES: Cc1ccnc(N)c1 ; O=C(CBr)c1ccccc1 ; Cc1ccn2cc(-c3ccccc3)nc2c1]

### Procedure for the Synthesis of 7-methyl-2-phenylimidazo[1,2-α]pyridine (BT-564):

A clean oven dried 25 mL round bottom flask was charged with 1 (1 equiv.), 2 (1.1 equiv.), NaHCO3 (3 equiv.) and ethanol (3 mL). The resulted reaction mixture was stirred at reflux condition for 8 h. Reaction progress was monitored by TLC. After completion, the reaction mass was allowed to cool at ambient temperature, diluted with water and extracted with EtOAc (2×10 mL). The combined organic layer was dried with anhydrous Na₂SO₄ and evaporated under reduced pressure. The crude material was purified by column chromatography on silica gel (100-200 mesh). Light pink solid (Yield =90%), ¹H NMR (400 MHZ, DMSO) δ 8.41 (d, J=6.9 Hz, 1H), 8.31 (s, 1H), 7.94 (d, J=7.2 Hz, 2H), 7.43 (t, J=7.6 Hz, 2H), 7.35 (s, 1H), 7.30 (t, J=7.3 Hz, 1H), 6.74 (dd, J=6.9, 1.2 Hz, 1H), 2.35 (s, 3H). ¹³C NMR (100 MHZ, DMSO) d 145.7, 144.6, 135.8, 134.6, 129.1, 128.0, 126.5, 1259, 115.4, 115.2, 109.0, 21.3.

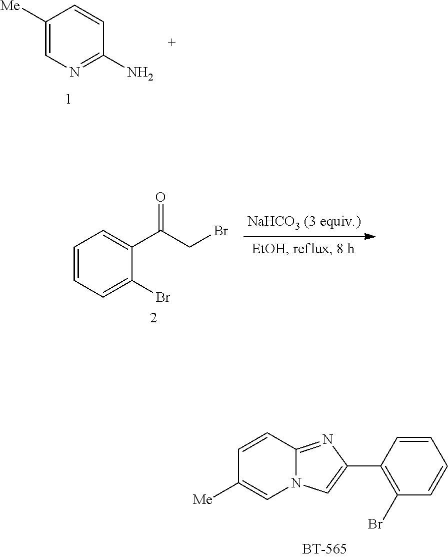 [SMILES: Cc1ccc(N)nc1 ; O=C(CBr)c1ccccc1Br ; Cc1ccc2nc(-c3ccccc3Br)cn2c1]

### Procedure for the Synthesis of 2-(2-bromophenyl)-7-methylimidazo[1,2-α]pyridine (BT-565);

A clean oven dried 25 mL round bottom flask was charged with 1 (1 equiv.), 2 (1.1 equiv.), NaHCO₃ (3 equiv.) and ethanol (3 mL). The resulted reaction mixture was stirred at reflux condition for 8 h. Reaction progress was monitored by TLC. After completion, the reaction mass was allowed to cool at ambient temperature, diluted with water and extracted with EtOAc (2×10 mL). The combined organic layer was dried with anhydrous Na₂SO₄ and evaporated under reduced pressure. The crude material was purified by column chromatography on silica gel (30% ethyl acetate: 70% hexane). yellow solid (Yield=80%), ¹H NMR (400 MHz, DMSO) δ 8.49 (s, 1H), 8.42 (s, 1H), 8.11 (d, J=7.5 Hz, 1H), 7.73 (d, J=7.8 Hz, 1H), 7.58-7.44 (m, 2H), 7.28 (t, J=7.2 Hz, 1H), 7.16 (d, J=9.1 Hz, 1H), 2.29 (s, 3H). ¹³C NMR (100 MHz, DMSO) 6 143.2, 142.1, 134.8, 134.1, 131.8, 129.6, 128.9.128.2, 124.9, 122.0, 121.0, 116.5, 112.4, 18.0.

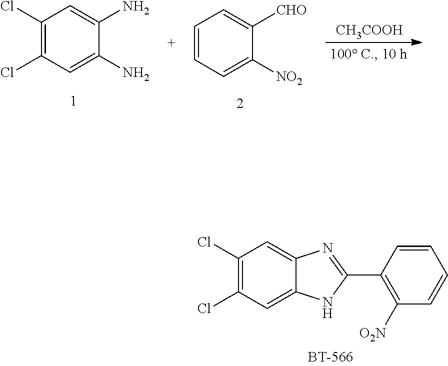

### Procedure for the Synthesis of 5,6-dichloro-2-(2-nitrophenyl)-1H-benzo[d]imidazole (BT-566);

A clean oven dried 25 mL round bottom flask was charged with 1 (1 equiv.), 2 (1.1 equiv.) and acetic acid (3 mL). The resulted reaction mixture was stirred at 100° C. for 10 h. Reaction progress was monitored by TLC. After completion, the reaction mass was allowed to cool at ambient temperature, reaction mixture was neutralized by NaHCO3, diluted with water and extracted with EtOAc (2×10 mL). The combined organic layer was dried with anhydrous Na₂SO₄ and evaporated under reduced pressure. The crude material was purified by column chromatography on silica gel (100-200 mesh).

### EXAMPLES

YAP depletion in vitro was used to screen for novel mediators of podocyte injury,

and KCa3.1, a calcium activated potassium channel, was identified as such. The calcium-activated potassium channel family is divided into three subtypes: large conductance or BK channels, intermediate conductance (IK channels, including KCa3.1) and small conductance SK channels. KCa3.1 channels promote potassium efflux that may result in cellular shrinkage and apoptosis (21). A functional role for KCa3.1 channels is the regulation of calcium entry into cells (22). KCa3.1 channel mediated potassium efflux may result in membrane hyperpolarization and cause increased cellular calcium entry through Ca²⁺ release-activated Ca²⁺ channels (CRAC) or transient receptor potential (TRP) channels. The calcium sensor calmodulin is tightly bound to the C-terminal domain of the homotetrameric KCa3.1 channels, resulting in high sensitivity to changes in intracellular calcium (23).

### Gene Silencing of YAP Disrupts the Podocyte Actin Cytoskeleton and Focal Contacts

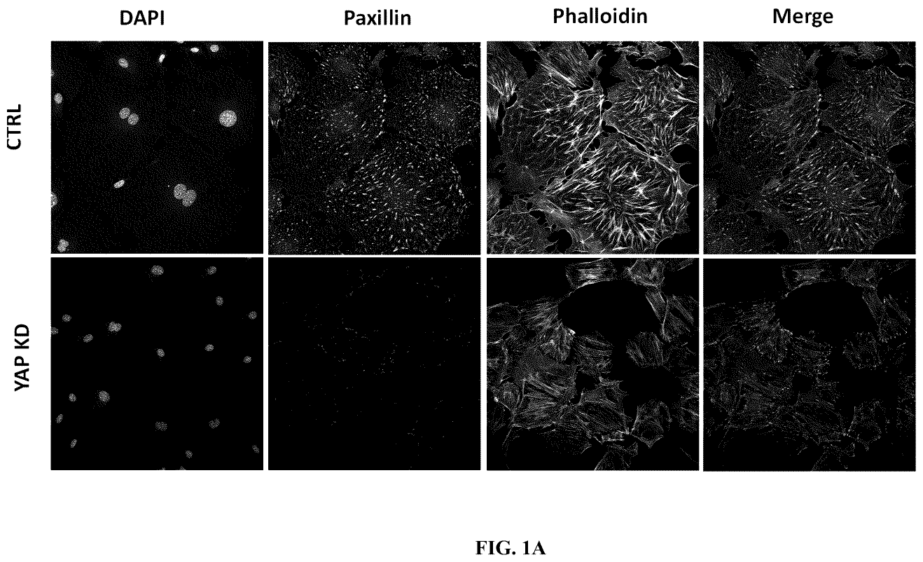

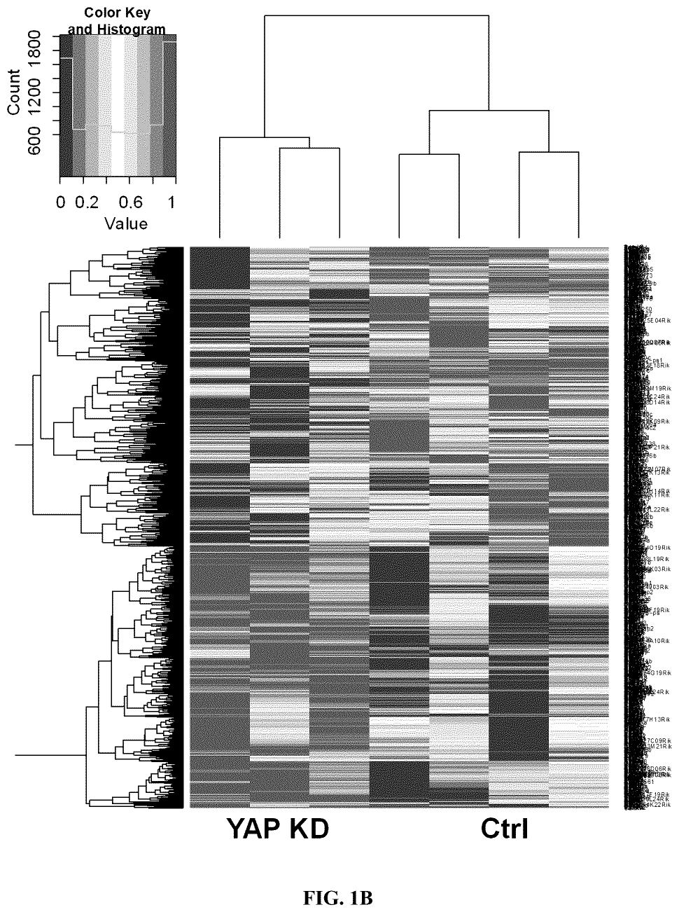

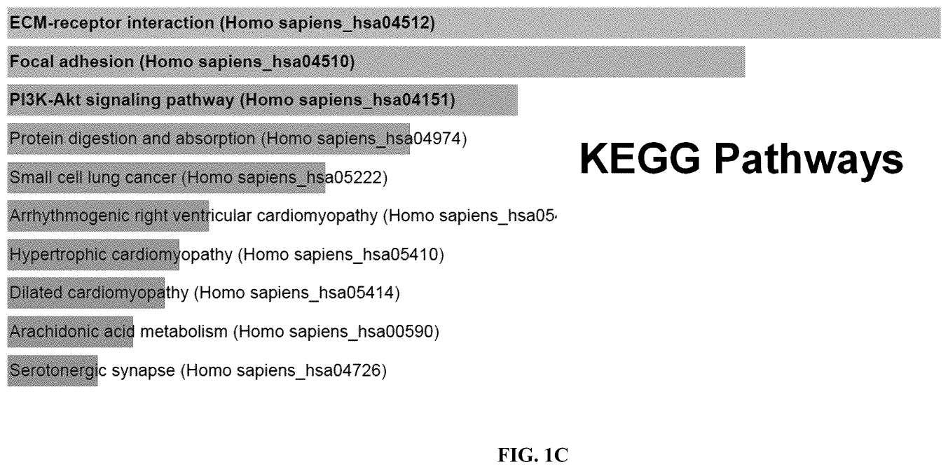

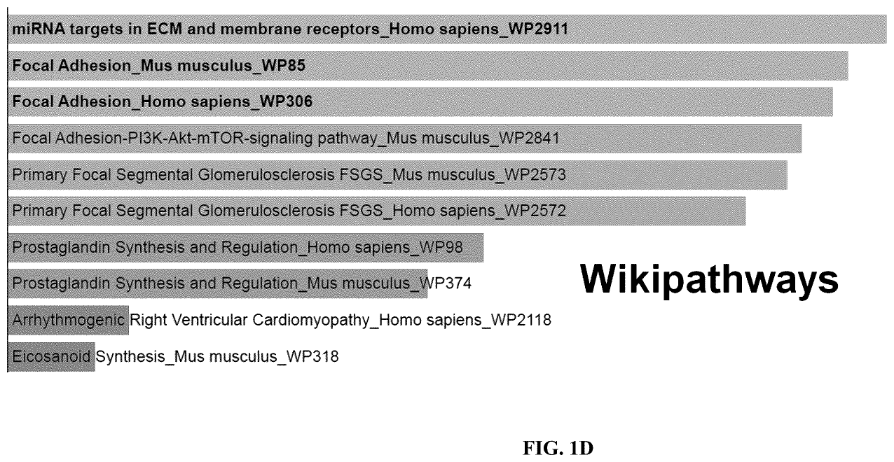

YAP silencing in podocytes in vitro enhances susceptibility to apoptosis and YAP silencing in podocytes in vivo is sufficient to cause focal segmental glomerulosclerosis (14, 15). YAP expression and activity is reduced in human FSGS (15, 16). YAP was silenced in immortalized mouse podocytes by lentiviral shRNA infection according to established protocols (14, 24). YAP knockdown podocytes showed a significant reduction in overall size as well as F-actin and focal adhesion marker expression compared to control podocytes (FIG. 1A). RNA-Seq was used to compare the transcriptomic profile of control and YAP knockdown (YAP KD) podocytes (FIG. 1B). Through KEGG and Wikipathway enrichment analysis, it was confirmed that the genes involved in cell-matrix and focal adhesion assembly are the most dysregulated with YAP silencing. In addition, the calcium-regulated PI3K-Akt signaling pathway was among the top perturbed in both KEGG and Wikipathway analysis (FIG. 1C and 1D).

### Network Analysis of RNA Seq Data

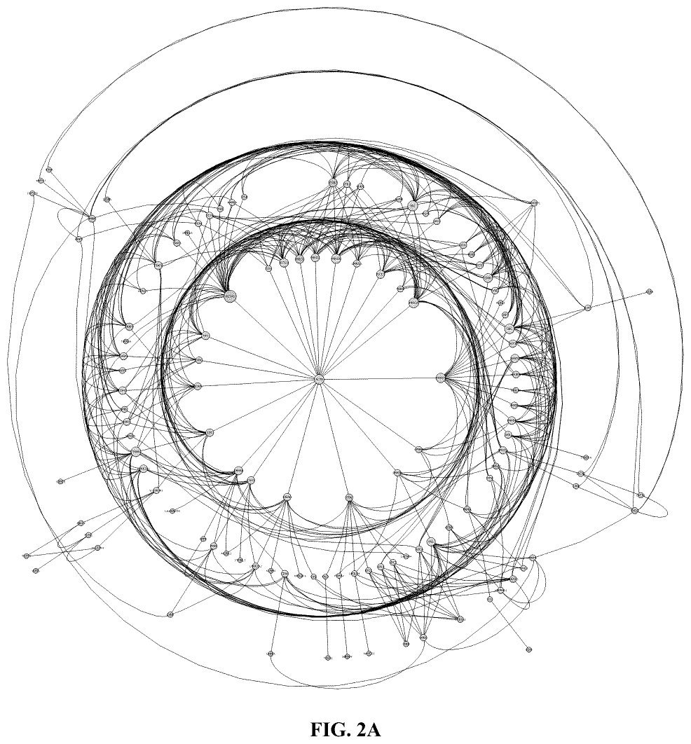

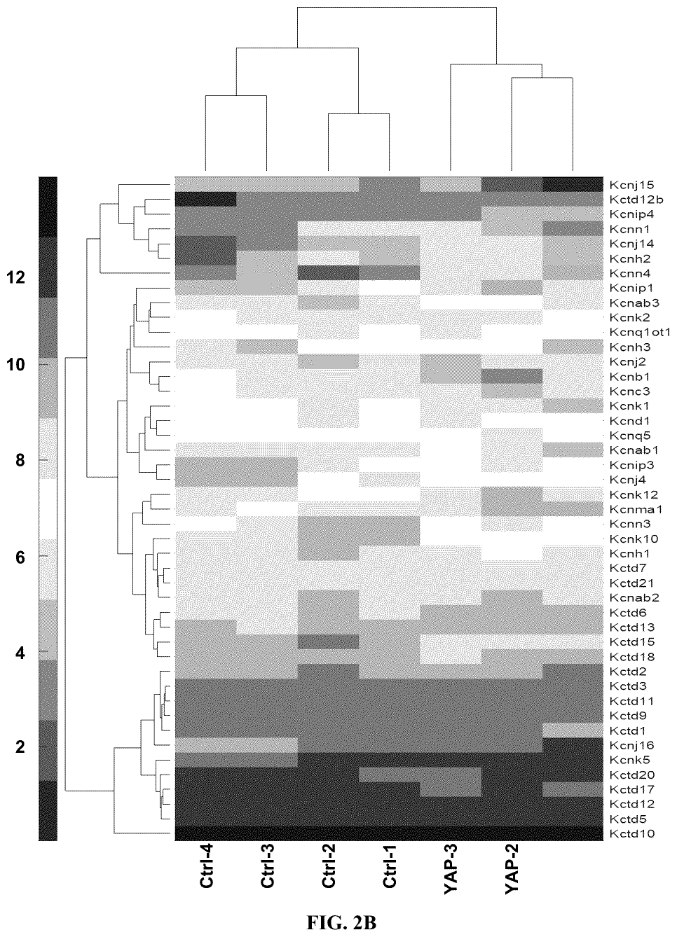

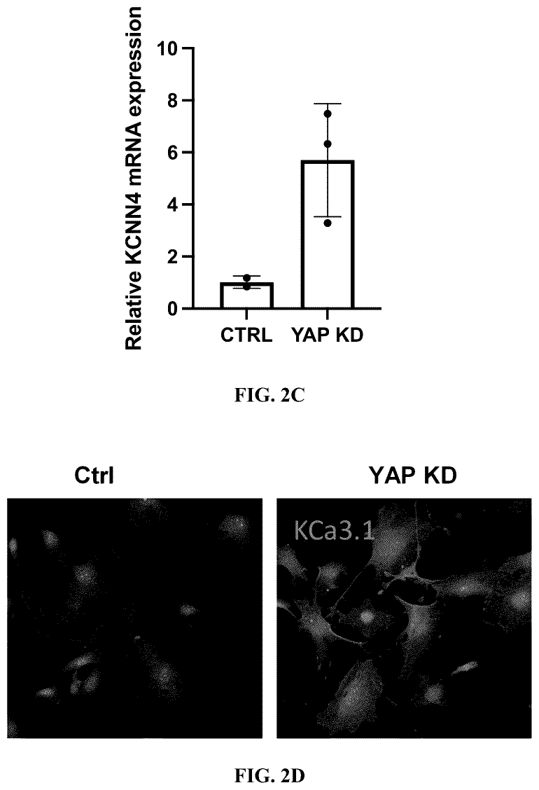

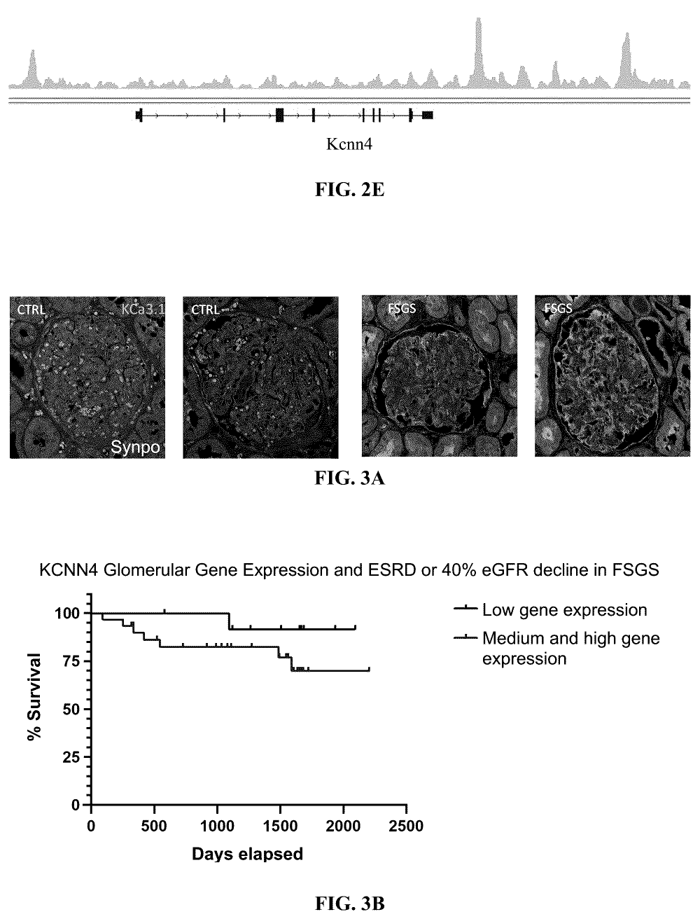

Network analysis was performed on the differentially expressed genes by cross-referencing nodal distance metrics of top genes across a curated human protein-protein interactome (25) using the nearest neighbors approach (26). The results were coherent with a singular interconnected network that included 122 nodes and 478 edges (FIG. 2A). The node with the highest centrality and connectivity was actin, further highlighting the consistency of the findings that actin-associated genes are affected by YAP modulation. From the differentially expressed RNA-Seq targets, TRANSFAC and JASPAR position weight matrix databases (27, 28) were used to generate a YAP-dependent podocyte specific upstream transcriptional profile signature. One of the top upregulated genes in the RNA Seq analysis of YAP silenced podocytes was KONN4 (21.14 fold increase, p-value 2.56E-17). KONN4 encodes the calcium activated potassium channel KCa3.1, previously shown to be pathogenic and a putative therapeutic target for tubulointerstitial injury in diabetic mice (29, 30). Integrated network analysis of the RNA-Seq experiment with WT and YAP KD podocytes was performed to evaluate the role of KONN4 in YAP mediated phenotypic homeostasis. The most significantly enriched gene ontology molecular function term among the differentially upregulated genes was “potassium channel regulator activity”. This ontology, and all the statistically significant ontological families within one step of KCNN4 (including Panther, GO, KEGG and Reactome), were manually curated. Many of the binding partners of KCa3.1 are affected by YAP KD. Furthermore, while KONN4 may be the most distinctly upregulated gene, it is not the singular differentially expressed component and genes responsible for potassium homeostasis may be broadly targeted in YAP KD (FIG. 2B). Expression of KONN4/KCa3.1 is increased at the transcriptional and protein level in YAP knockdown podocytes, shown by qPCR (FIG. 2C) and immunofluorescence imaging (FIG. 2D), respectively. Expression of the encoded KCa3.1 channel was seen much more clearly at the cell membrane (where it would be expected to be more functionally active) with YAP silencing (FIG. 2D). YAP has been reported to be a transcriptional co-repressor for KCNN4 in MCF10A breast epithelial cells (31). Chromatin immunoprecipitation was performed followed by next-generation sequencing (ChIP-seq) with an anti-YAP antibody in differentiated mouse podocytes. YAP localizes on multiple sites near the KONN4 gene in podocytes with notable exclusion from its promoter, consistent with binding to silencers (FIG. 2E). This suggests that in podocytes YAP may similarly act as a repressor of KCNN4 gene expression.

### KCNN4 Expression in Human Glomerular Disease

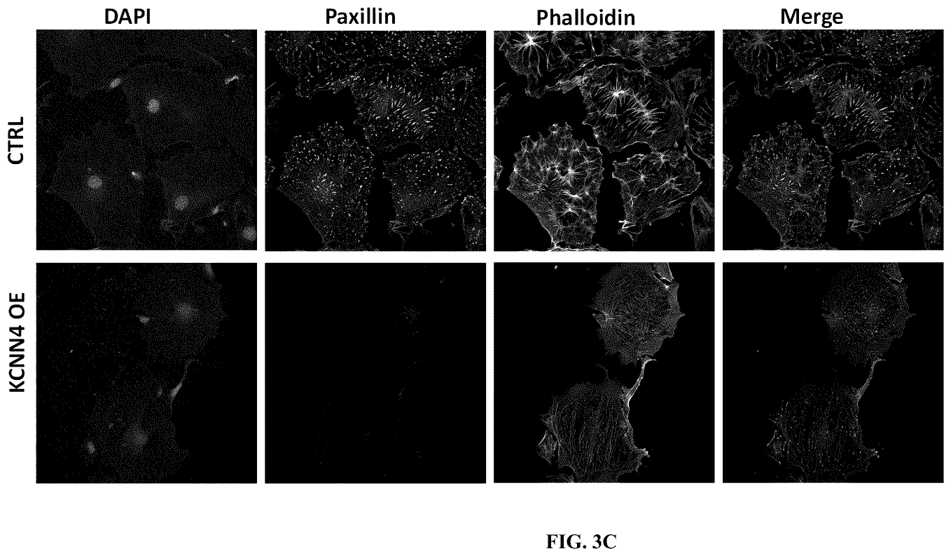

Expression of KCNN4 in human glomerular disease was determined by double labeling immunofluorescence with synaptopodin as a podocyte marker on tissue from patients with FSGS, a clinical podocytopathy. At baseline, much like in wild type murine podocytes (FIG. 2D), KCa3.1 expression was low in control patients with no glomerular disease. By contrast, KCa3.1 glomerular expression was increased in individuals with biopsy-proven FSGS (FIG. 3A). Using data from the NIH-funded NEPTUNE consortium, the correlation between glomerular KCNN4 gene expression and clinically relevant renal parameters was evaluated. For patients with biopsy-proven FSGS, during an average follow up of 35.4 months, higher KCNN4 glomerular expression was significantly associated with increased risk of reaching the composite endpoint of end stage renal disease (ESRD) or 40% decline in estimated glomerular filtration rate using Cox's regression model. Higher KCNN4 glomerular expression was significantly associated with increased risk of reaching the composite endpoint (FIG. 3B). Similar to the injury conferred by YAP silencing, compared to controls, KCNN4 OE podocytes exhibited diminished focal adhesion marker expression and disorganized actin cytoskeleton determined by paxillin and phalloidin expression respectively (FIG. 3C).

### Development of Novel KCa3.1 Inhibitors

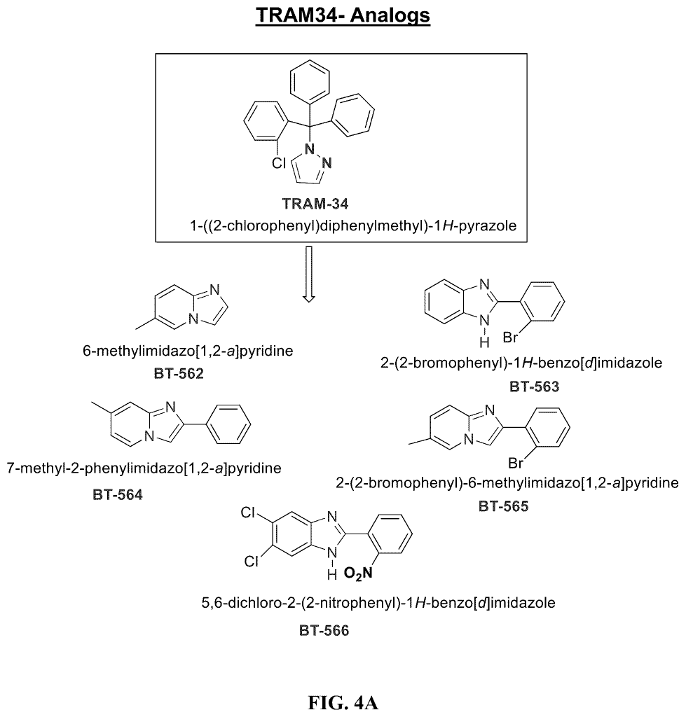

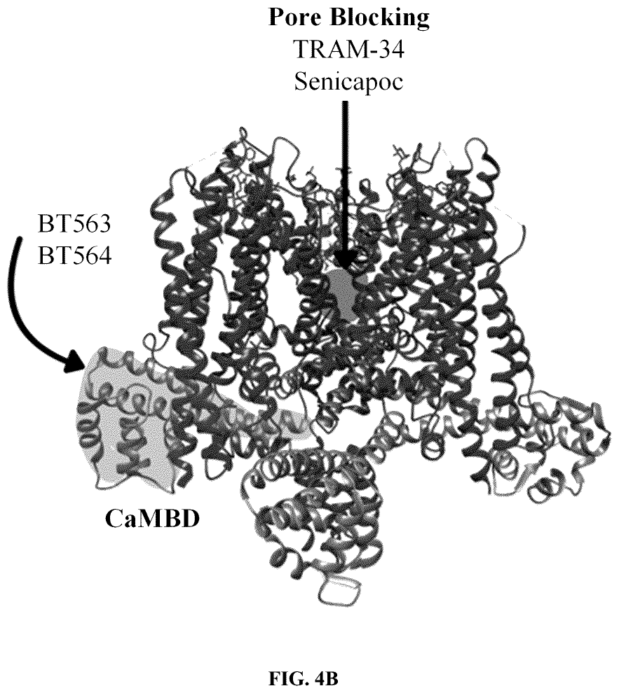

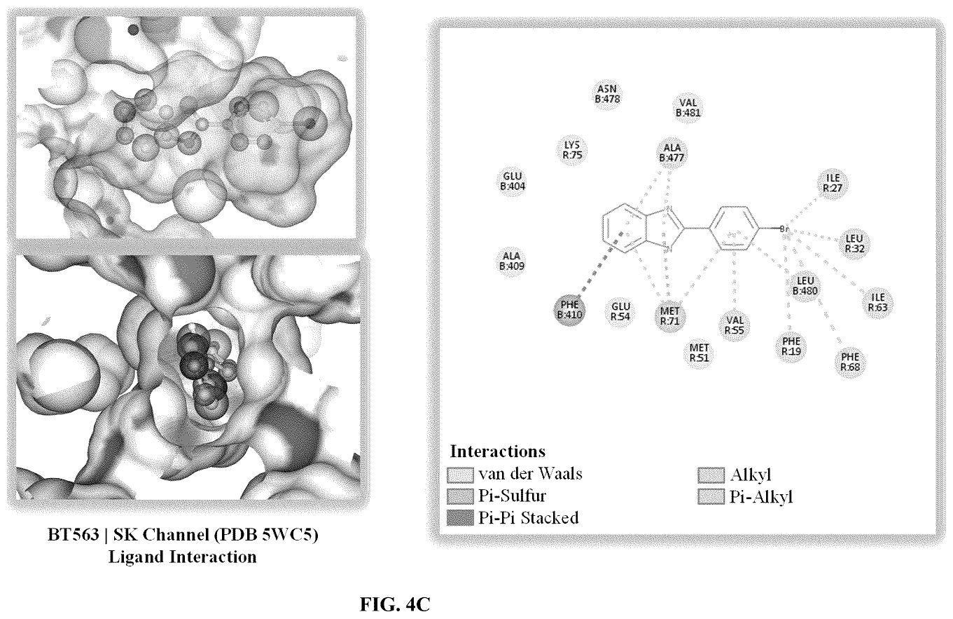

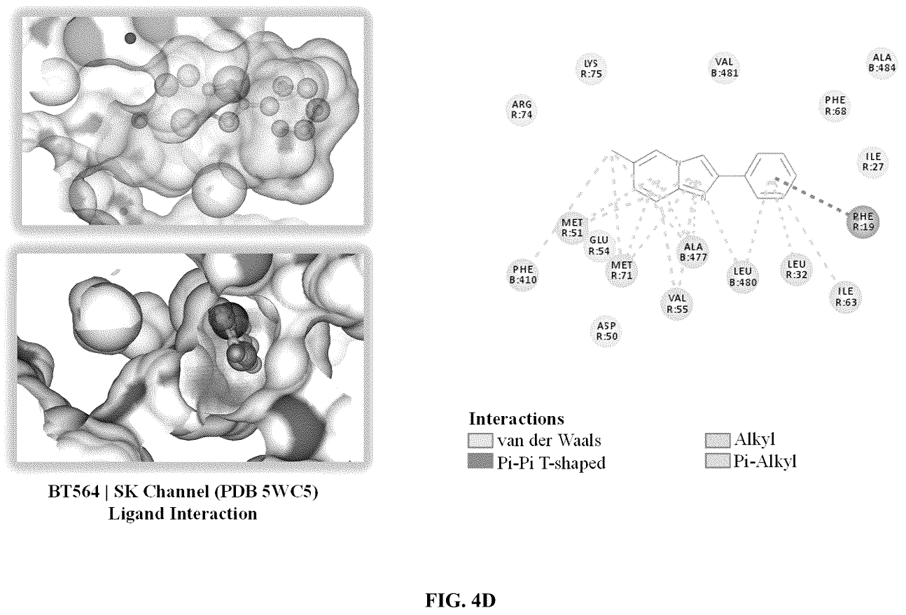

Chemical synthesis was complemented by structure based virtual screening using SeeSar (BioSolveIT, Germany). TRAM-34 and Senicapoc are two precedent bioactive small molecules that block KCa3.1 currents by binding to the inner pore of the channel, thereby obstructing the flow of K ions (32). A second, smaller allosteric pocket adjacent to the intracellular calmodulin binding domain (CaMBD) is located at the proximal carboxyl-terminus of the KCa3.1 channel, identified as a site of action for previously characterized positive channel modulators (33, 34). Ten (10) compounds were initially synthesized to evaluate based on desired biological activity. The virtual screening approach revealed that five (5) of the initial compounds (BT562, BT563, BT564, BT565, BT566)were able to viably fit inside the smaller allosteric pocket but were too small to form favorable interactions with the residues inside the channel to effectively block the pore (FIGS. 4B-4D).

### KCa3.1 Inhibition Protects Podocytes From Injury

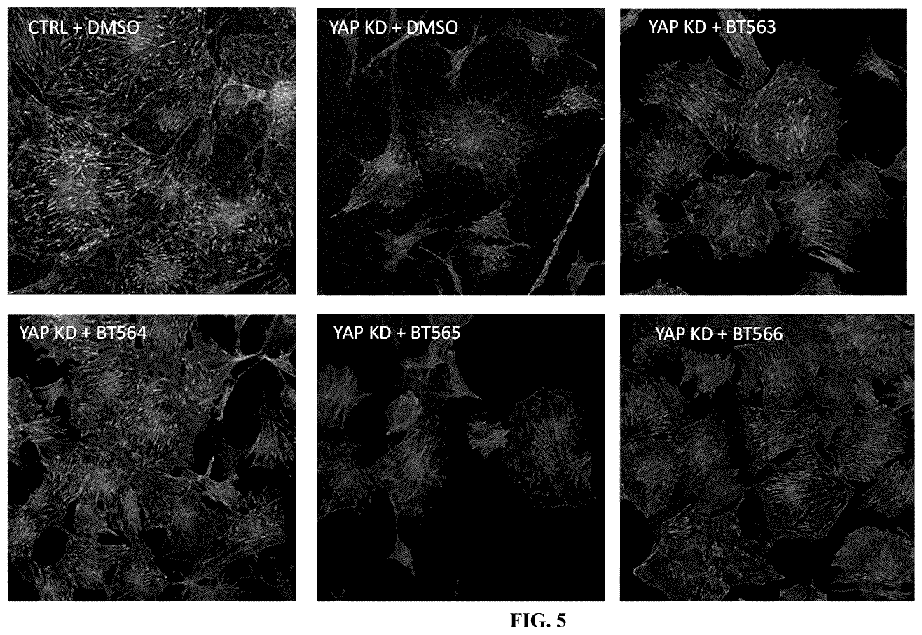

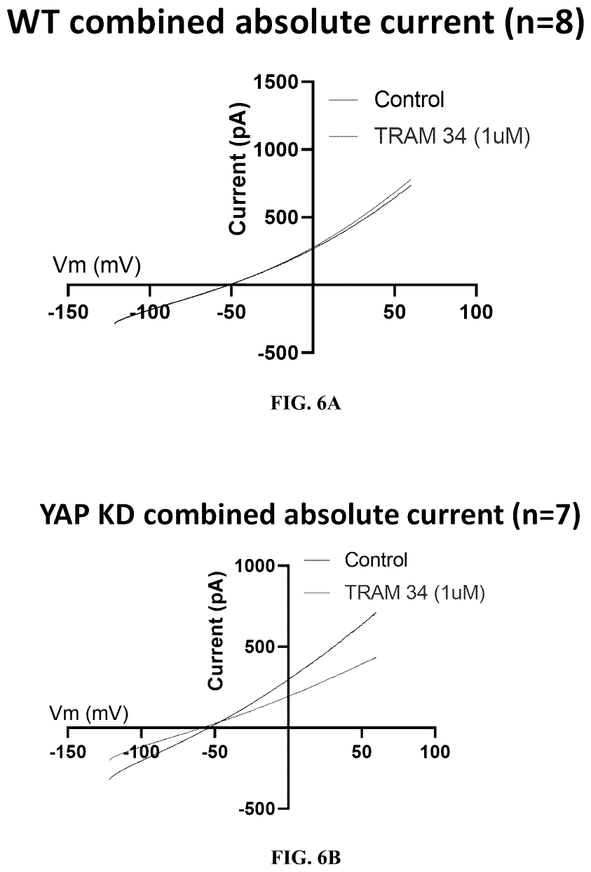

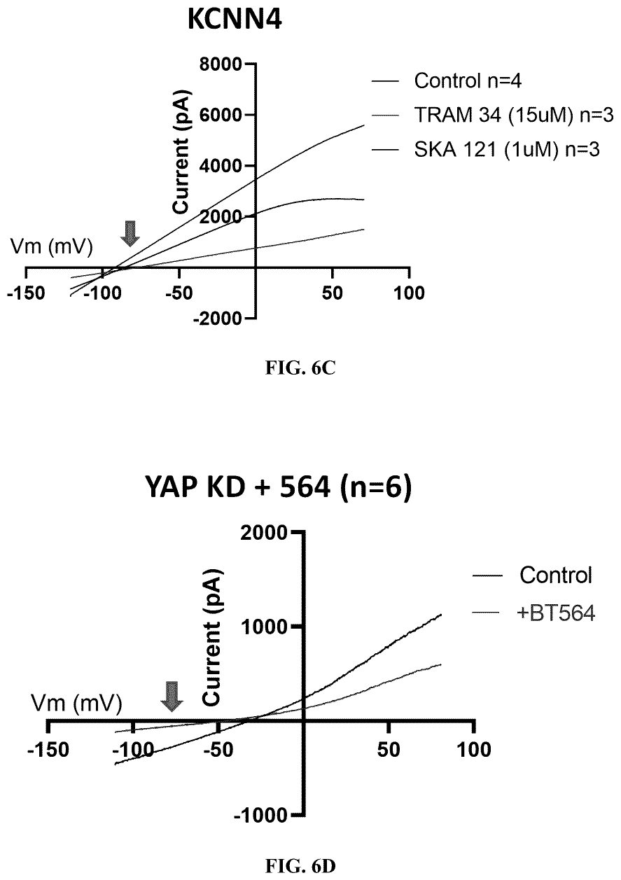

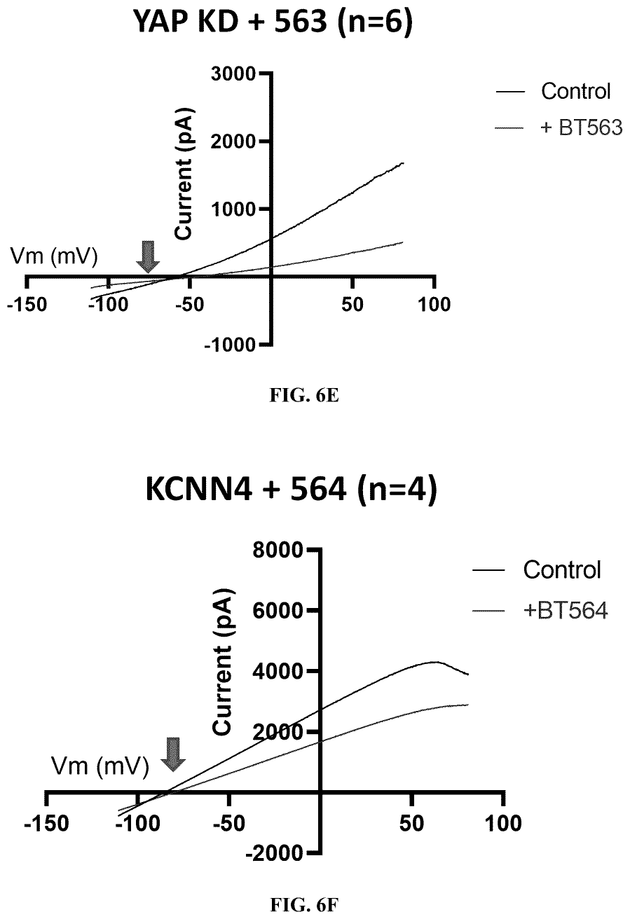

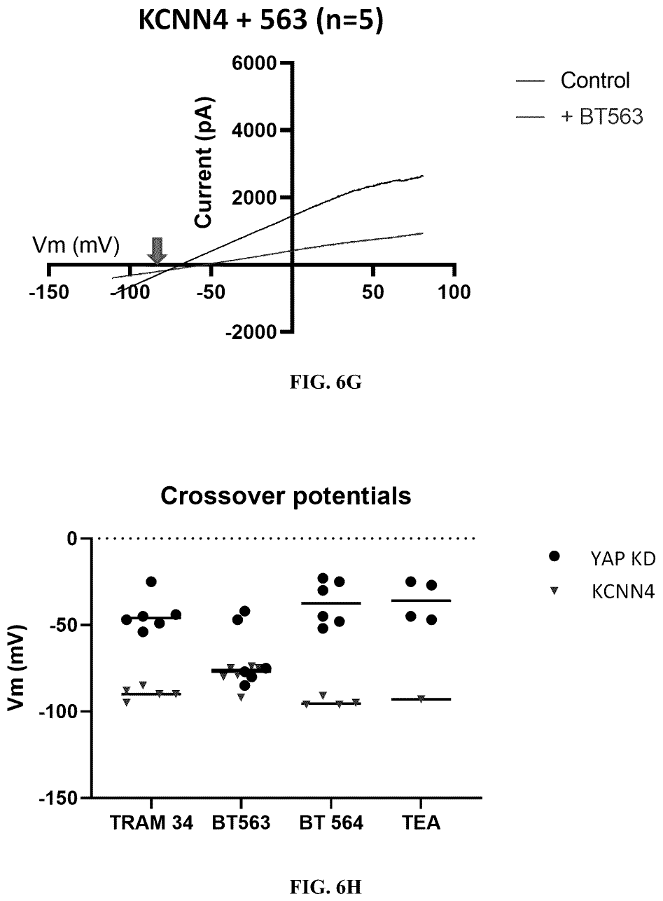

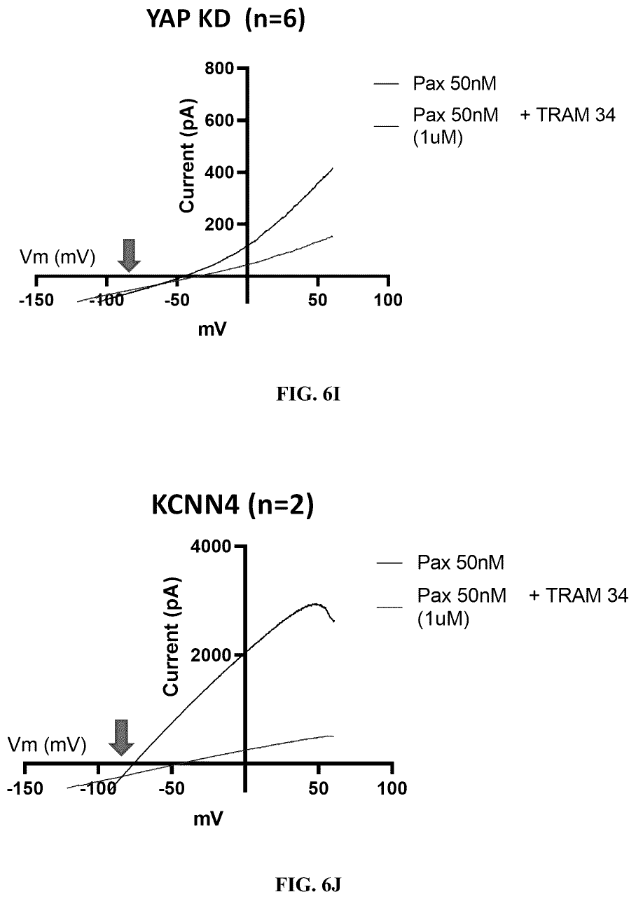

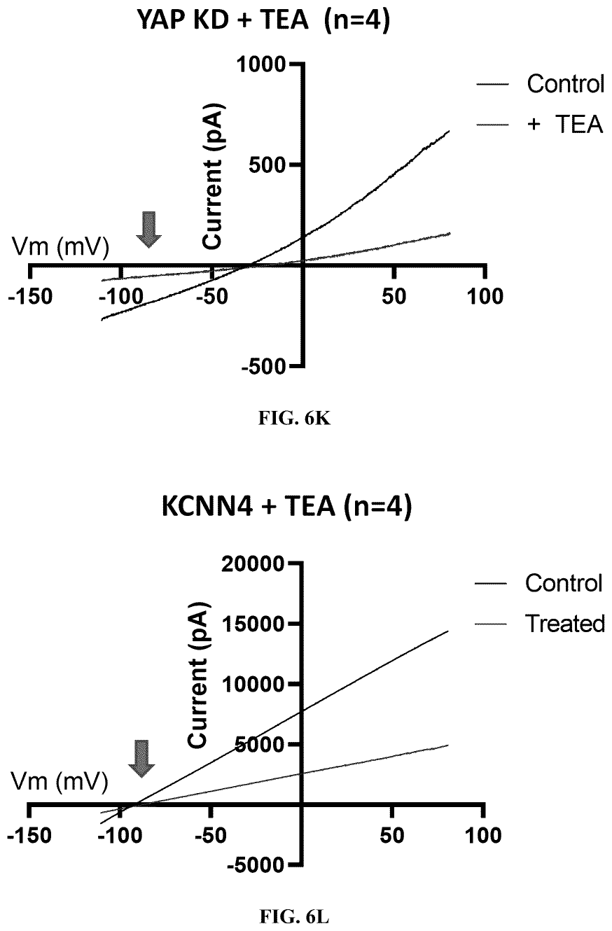

The five (5) compounds (BT562 through BT566)were tested to determine whether they protect YAP KD podocytes from injury. Novel KCa3.1 inhibitors BT563 and BT564 most consistently rescued YAP knockdown podocytes from focal adhesion loss and F-actin reorganization (FIG. 5). Patch clamp electrophysiology with wild type, YAP knockdown and KCNN4 overexpression podocytes in the presence and absence of the KCa3.1 activator SKA121 as well as KCa3.1 inhibitors TRAM-34, BT563 and BT564 was performed. YAP silenced as well as KCNN4 OE podocytes had increased KCa3.1 conductance when compared to wild type cells that was enhanced by SKA121 and decreased with TRAM34 (FIGS. 6A-6C). The before and after averaged I-V TRAM34 plots crossed at —50 mV in YAP silenced cells and not at the expected K+equilibrium (VK) calculated at —83 mV. Similar results were observed in YAP silenced cells with senicapoc treatment (not shown), as well as with paxilline (a BK channel blocker) (FIGS. 6I and 6J) and the nonselective potassium channel blocker TEA (tetraethylammonium) (FIGS. 6K and 6L). TRAM-34 plots did cross at the expected K+ equilibrium in KCNN4 OE podocytes. BT563 and BT564 also decreased KCa3.1 conductance in KCNN4 OE and YAP silenced podocytes (FIGS. 6D-6G). BT564 behaved much like TRAM-34, TEA and paxilline with a crossover potential as expected of —85 in KCNN4 OE cells but around —50 in YAP knockdown cells. BT563 was the only compound tested (including those commercially available) that had a crossover potential closer to the expected K+ equilibrium (VK) with treatment of YAP silenced podocytes.

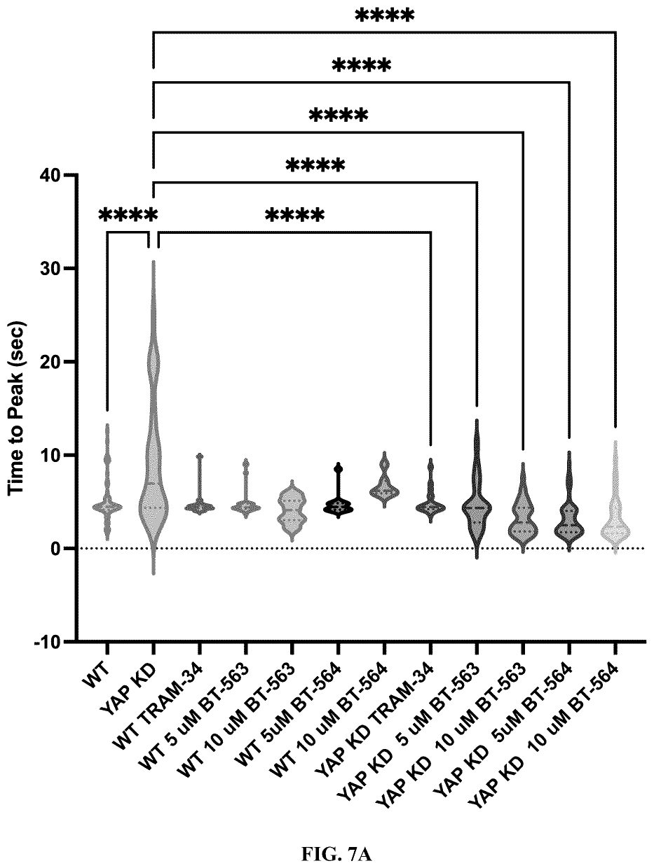

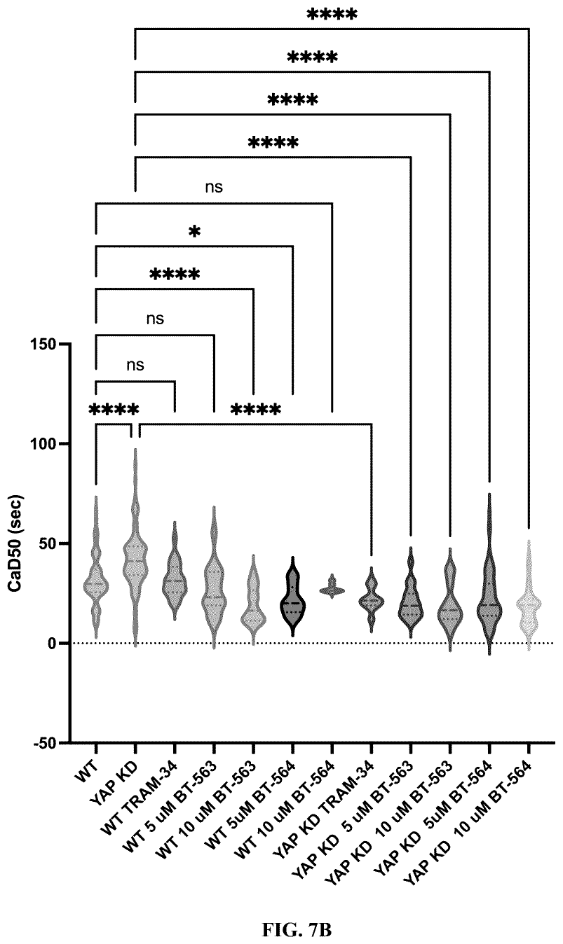

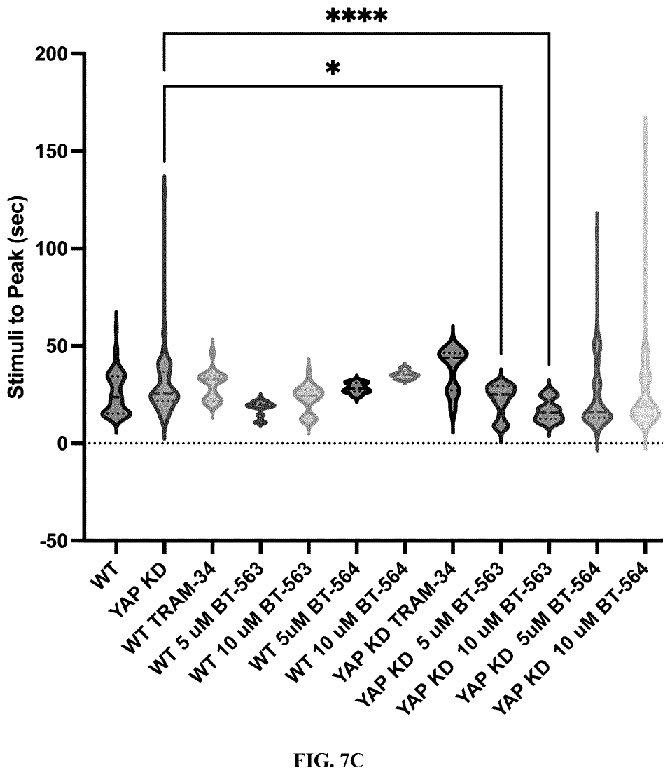

Intracellular calcium dynamics were measured in wild type and YAP KD podocytes loaded with Fluo-3, stimulated with ATP (500 mM) and imaged for 3 minutes at 1 frame per 0.5 seconds on a high-speed Zeiss 880 laser scanning confocal microscope with automated CO₂incubator stage. The time to peak of the calcium uptake and the CaD50, duration between 50% or less of the maximum amplitude during the transient period of influx calcium, in response to ATP stimulation was prolonged by knocking down YAP in podocytes. When treated with KCa3.1 inhibitors such as TRAM-34, BT563 and BT564, YAP silenced podocytes had a decreased time to peak of calcium uptake and CaD50 upon ATP stimulation (FIGS. 7A-7C). Only BT563 (5uM and 10 uM) significantly decreased the stimuli to peak in the YAP knockdown cells.

### KCa3.1 Inhibition In Vivo

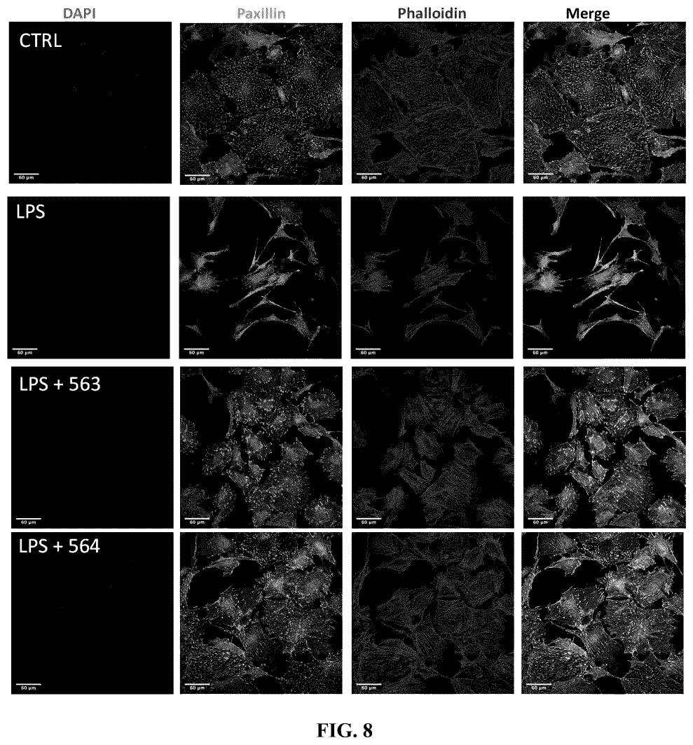

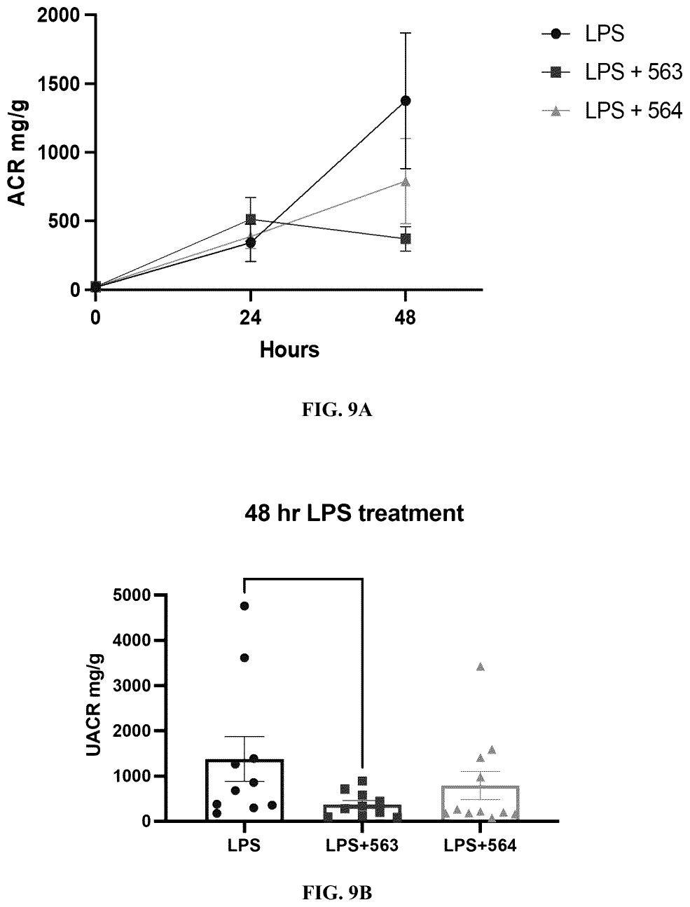

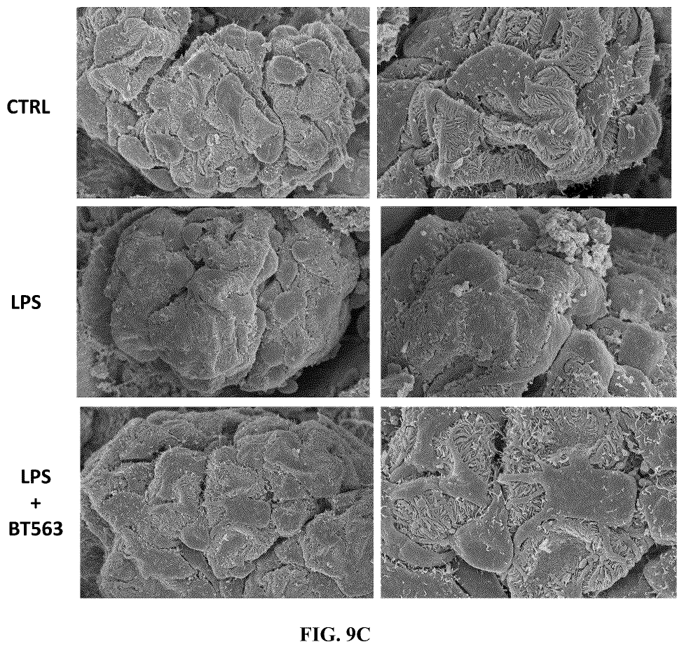

The ability of KCa3.1 inhibitors to protect from podocyte injury induced by lipopolysaccharide (LPS) was tested in vitro and in vivo. In vitro data showed that LPS (100 μg/ml for 24 hrs) treatment of podocytes induced actin cytoskeletal reorganization and loss of focal adhesions. These changes were abrogated by co-treatment with BT563 and BT564 (FIG. 8). The protective effect was tested in vivo by administering LPS to 8-week old C₅₇BL/6J mice at 0 hr and 24 hrs. At 12 hr and 24 hr after the initial LPS injection, BT563 and BT564 were administered to these animals. LPS injected mice had a steady increase in albuminuria at 24 hrs and 48 hrs which was not seen in the PBS injected mice. Mice that were treated with BT563 had a statistically significant decrease in albuminuria at 48 hours after LPS injection (FIGS. 9A and 9B). Scanning EM images showed foot process injury with LPS treatment not seen with PBS injected controls or BT563 treated mice (FIG. 9C).

### METHODS

### Antibodies

The following antibodies were used: mouse anti-paxillin, clone 165 (BD

Biosciences), Rabbit anti-YAP (Novus NB110-58358), KCNN4 (Alomone), Alexa Fluor 488 goat anti-rabbit IgG (Life Technologies), phallodin-rhodamine (Life Technologies), Alexa Fluor 594 goat anti-mouse IgG (Life Technologies).

### Cell Culture

Podocytes were cultured as previously described (2, 14, 35). Briefly, conditionally immortalized murine podocytes were cultured in Dulbecco's modified Eagle's medium (DMEM; Life Technologies) supplemented with 10% heat-inactivated fetal bovine serum (Life Technologies) and 100 U/mL penicillin-streptomycin (Life Technologies) on type I collagen (Corning, Corning, NY) coated dishes or flasks. Undifferentiated podocytes were maintained at 33° C. in the presence of 10 U/mL mouse γ-interferon (Cell Sciences, Canton, MA) to drive T-antigen expression. To induce differentiation, podocytes were thermo-shifted to a 37° C. incubator and switched to medium lacking γ-interferon.

### Gene Silencing of YAP

pLKO.1 lentiviral shRNA plasmids were purchased from Addgene for scramble control and from Sigma-Aldrich for YAP. We used sequence:

5′-CCGGCGGTTGAAACAACAGGAATTACTCGAGTAATTCCTGTTGTTTCAACCGTTTTT G-3′ (SEQ ID NO: 1) to target YAP. The pLKO.1 plasmids (YAP shRNA or control shRNA) along with the helper plasmids psPAX² and pMD2.G were transfected into HEK293T cells at 70% confluence using FuGENE 6 (Promega). Medium was replaced 16-18 h after transfection. 36 and 60 h after transfection, virus-containing supernatants were harvested and centrifuged at 3,000 rpm for 5 min. Viral particles were then passed through a 0.45-μm filter. The supernatants were subsequently used for the infection of target cells in the presence of 4 μg/ml Polybrene (Sigma). Wild-type undifferentiated podocytes were infected for 24 h and then selected. Noninfected podocytes cells were removed by selection in 5 μg/ml puromycin (Sigma). Podocytes were selected for ˜1 week, after which 1 μg/ml puromycin was used as the maintenance dose.

### Immunofluorescence Microscopy for KCNN4 in Native Human Renal Biopsies

Archival frozen renal biopsy cores were obtained by the renal biorepository at Icahn School of Medicine at Mount Sinai.

Deidentified, formalin-fixed, paraffin-embedded human tissues were cut in 4 um sections. Antigen retrieval was performed after deparaffinizing and rehydrating the sections. The slides were immersed in citrate buffer (10 mM citric acid, 0.05% Tween 20, pH 6.0), heated to boiling point for 5 min, cooled to room temperature for 20 min, washed in PBS two times, and air-dried for 10 min at room temperature. Sections were then stained with primary antibodies against KCNN4 and synaptopodin overnight at 4C, washed with PBS, and then incubated with Alexa Flour secondary antibodies (Life Technologies) for 1 hour. All images were then taken with a confocal microscope.

### LPS Treatment of Cultured Podocytes

Differentiated podocytes were exposed to different doses of LPS (50-100 μg/ml) for 24 hours. Changes in the organization of the actin cytoskeleton were assessed by phalloidin staining and paxillin staining as described previously. Each set of experiments was repeated at least three times. Briefly, cells were fixed with 4% PFA, 4% sucrose in PBS at room temperature for 15 minutes and then permeabilized with 0.3% Triton X-100 in PBS with washes in between. Coverslips were washed 3× with PBS and incubated blocking solution (2% FCS, 2% BSA, 0.2% fish gelatin) for 30 minutes at room temperature before further incubation with one of the primary antibodies for 1 hour at room temperature. For double labeling, sections were washed three times with PBS for 5 minutes, and the second primary antibody was applied for 60 minutes. Antigen-antibody complexes were visualized with secondary antibodies 1:500 (Life Technologies) conjugated with fluorochrome (Alexa 488, Alexa 594, or Alexa 647) for 1 hour at room temperature. For stress fibers, phalloidin-rhodamine was used. For the nuclei, we used DAPI. Confocal microscopy with Leica DMI was done.

### Proteinuria Measurements

Albuminuria was qualitatively screened using a 10% SDS-PAGE gel followed by Coomassie-blue staining. BSA standards of 0.1, 0.5, 5 μg were used and 5 μl urine was used from each sample to quantify urine albumin in duplicate using commercial kits, according to the manufacturer's protocol (mouse albumin ELISA, Bethyl Laboratories).

### Whole-Cell Patch Recordings

Cells plated on coverslips are transferred to a submersion recording chamber and continuously perfused (2-4 ml/min) with RPMI cell culture medium containing (in mM): NaCl 124, KCl 2.5, NaH₂PO₄ 1.2, NaHCO₃ 24, HEPES 5, glucose 12.5, MgSO₄ 2, and CaCl₂ 2, pH 7.4. The solution is continuously bubbled with 95% O₂/5% CO₂, and recordings are obtained at 25±1° C. Cells are recorded under infrared differential contrast optics (BX51; Olympus). After control readings are measured, the solution is then switched to the solutions containing the molecules. Cells are recorded under infrared differential contrast optics (BX^{51;} Olympus). Recording pipettes (3-5 MΩ), pulled from borosilicate glass, are filled with internal solution containing (in mM): K gluconate 120, HEPES 10, KCl 20, MgCl2 1, MgATP 2, Na2GTP 0.5, Na-phosphocreatine 10, CaCl2 1.71 and EGTA 2, pH 7.25. Data are acquired with a Multiclamp 700 A amplifier (Molecular Devices), subsequently filtered at 10 kHz and digitized at 10 kHz, and analysis is performed with pClamp 11 software (Molecular Devices). Whole-cell data will be collected. All data will be recorded at room temperature.

### RNA-Sequencing

Bulk RNA-sequencing was performed on 4 control knockdown and 9 YAP knockdown murine podocytes. Total RNA was extracted using TRIzol (Thermo Fisher Scientific). The RNA quality was evaluated by an Agilent 2100 Bioanalyzer. The cDNA library preparation (RNA with PolyA selection) and sequencing was performed at Genewiz/Azenta using an Illumina HiSeq system. The reads with good quality were first aligned to mouse reference databases including mouse genome, exon, and splicing junction segment and a contamination database including ribosome and mitochondria sequences using the STAR alignment algorithm. After filtering reads mapped to the contamination database, the reads that were uniquely aligned to the exon and splicing junction segments with a maximal two mismatches for each transcript were then counted as the expression level for the corresponding transcript.

### Calcium Dynamics: Confocal Recording and Solutions

Isolated cells were superfused with Tyrode solution containing (mm): NaCl 140, KCl

5, Hepes 5, NaH₂PO₄ 1, MgCl₂ 1, CaCl₂ 1.8, glucose 10 (pH 7.4). To allow for confocal imaging of [Ca²⁺]ᵢ, cells were loaded for 30 min with 5 μm fluo-3 AM (Molecular Probes, Eugene, OR, USA), then washed and stored for 20 min in Tyrode solution to enable dye de-esterification. Confocal imaging in line-scan mode was performed on a Zeiss 510 microscope. Cells were scanned with light at 488 nm from an argon-ion laser, and fluorescence above 505 nm was recorded. To generate repeated Ca²⁺ sparks at a limited number of locations, 50 nm ryanodine (Calbiochem, San Diego, CA, USA) was added to the external solution.

### Virtual Screening

A library of analogs was virtually screened against the two potential binding pockets associated with KCa3.1 channels. Two Protein Data Bank (PDB) files (allosteric: 5WC₅, channel: 6CNM) were cleaned and prepared in BioSolveIT SeeSAR. The novel analogs were prepared for docking by converting them from SMILES to SDF chemical-data. file format (SeeSAR version 12.0.1; BioSolveIT GmbH, Sankt Augustin, Germany). The ligands were placed and fitted into the binding pockets using an incremental construction algorithm available through the FlexX docking functionality in SeeSAR. A total of 500 poses were generated for each ligand. Representative free energies based on physical interactions and desolation of the ligands were calculated through the HYdrogen bond and DEsolvation (HYDE) algorithm which is a readily available feature in SeeSAR. The binding affinities for all poses were ranked and prioritized through the HYDE scoring function. The final candidates for structure-activity relationship (SAR) optimization was based on the top 3 poses for each ligand with the highest estimated affinity, best torsional quality, and lowest intermolecular and intramolecular clash scores.

### REFERENCES

1. Asanuma K, and Mundel P. The role of podocytes in glomerular pathobiology. Clin Exp Nephrol. 2003; 7(4):255-9.
2. Shankland SJ, Pippin JW, Reiser J, and Mundel P. Podocytes in culture: past, present, and future. Kidney Int. 2007; 72(1):26-36.
3. Greka A, and Mundel P. Cell biology and pathology of podocytes. Annu Rev Physiol. 2012; 74:299-323.
4. Wiggins RC. The spectrum of podocytopathies: a unifying view of glomerular diseases. Kidney Int. 2007; 71(12):1205-14.
5. Wharram BL, Goyal M, Wiggins JE, Sanden SK, Hussain S, Filipiak WE, et al. Podocyte depletion causes glomerulosclerosis: diphtheria toxin-induced podocyte depletion in rats expressing human diphtheria toxin receptor transgene. J Am Soc Nephrol. 2005; 16(10):2941-52.
6. Greka A, and Mundel P. Calcium regulates podocyte actin dynamics. Semin Nephrol. 2012; 32(4):319-26.

7. Burford JL, Villanueva K, Lam L, Riquier-Brison A, Hackl MJ, Pippin J, et al. Intravital imaging of podocyte calcium in glomerular injury and disease. J Clin Invest. 2014; 124(5):2050-8.
8. Reiser J, Gupta V, and Kistler AD. Toward the development of podocyte-specific drugs. Kidney Int. 2010; 77(8):662-8.
9. Leeuwis JW, Nguyen TQ, Dendooven A, Kok RJ, and Goldschmeding R. Targeting podocyte-associated diseases. Adv Drug Deliv Rev. 2010; 62(14):1325-36.
10. Cai J, Zhang N, Zheng Y, de Wilde RF, Maitra A, and Pan D. The Hippo signaling pathway restricts the oncogenic potential of an intestinal regeneration program. Genes Dev. 24(21):2383-8.
11. Huang J, Wu S, Barrera J, Matthews K, and Pan D. The Hippo signaling pathway coordinately regulates cell proliferation and apoptosis by inactivating Yorkie, the Drosophila Homolog of YAP. Cell. 2005; 122(3):421-34.
12. Sudol M, Shields DC, and Farooq A. Structures of YAP protein domains reveal promising targets for development of new cancer drugs. Semin Cell Dev Biol. 2012; 23(7):827-33.
13. Johnson R, and Halder G. The two faces of Hippo: targeting the Hippo pathway for regenerative medicine and cancer treatment. Nat Rev Drug Discov. 2014; 13(1):63-79.
14. Campbell KN, Wong JS, Gupta R, Asanuma K, Sudol M, He JC, et al. Yes-associated protein (YAP) promotes cell survival by inhibiting proapoptotic dendrin signaling. J Biol Chem. 2013; 288(24):17057-62.
15. Schwartzman M, Reginensi A, Wong JS, Basgen JM, Meliambro K, Nicholas SB, et al. Podocyte-Specific Deletion of Yes-Associated Protein Causes FSGS and Progressive Renal Failure. J Am Soc Nephrol. 2015.
16. Meliambro K, Wong JS, Ray J, Calizo RC, Towne S, Cole B, et al. The Hippo pathway regulator KIBRA promotes podocyte injury by inhibiting YAP signaling and disrupting actin cytoskeletal dynamics. J Biol Chem. 2017.
17. Wennmann DO, Vollenbroker B, Eckart AK, Bonse J, Erdmann F, Wolters DA, et al. The Hippo pathway is controlled by Angiotensin II signaling and its reactivation induces apoptosis in podocytes. Cell Death Dis. 2014; 5:e1519.
18. Bonse J, Wennmann DO, Kremerskothen J, Weide T, Michgehl U, Pavenstadt H, et al. Nuclear YAP localization as a key regulator of podocyte function. Cell Death Dis. 2018; 9(9):850.
19. Chen J, and Harris RC. Interaction of the EGF Receptor and the Hippo Pathway in the Diabetic Kidney. J Am Soc Nephrol. 2015.
20. Calizo RC, Bhattacharya S, van Hasselt JGC, Wei C, Wong JS, Wiener RJ, et al. Disruption of podocyte cytoskeletal biomechanics by dasatinib leads to nephrotoxicity. Nat Commun. 2019; 10(1):2061.
21. Lang PA, Kaiser S, Myssina S, Wieder T, Lang F, and Huber SM. Role of Ca2+-activated K+ channels in human erythrocyte apoptosis. Am J Physiol Cell Physiol. 2003; 285(6):C₁₅₅₃-60.
22. Bi D, Toyama K, Lemaitre V, Takai J, Fan F, Jenkins DP, et al. The intermediate conductance calcium-activated potassium channel KCa3.1 regulates vascular smooth muscle cell proliferation via controlling calcium-dependent signaling. J Biol Chem. 2013; 288(22):15843-53.
23. Wulff H, and Castle NA. Therapeutic potential of KCa3.1 blockers: recent advances and promising trends. Expert Rev Clin Pharmacol. 2010; 3(3):385-96.
24. Asanuma K, Campbell KN, Kim K, Faul C, and Mundel P. Nuclear relocation of the nephrin and CD2AP-binding protein dendrin promotes apoptosis of podocytes. Proc Natl Acad Sci USA. 2007; 104(24):10134-9.
25. Chen EY, Xu H, Gordonov S, Lim MP, Perkins MH, and Ma'ayan A. Expression2Kinases: mRNA profiling linked to multiple upstream regulatory layers. Bioinformatics. 2012; 28(1):105-11.
26. Berger SI, Posner JM, and Ma'ayan A. Genes2Networks: connecting lists of gene symbols using mammalian protein interactions databases. BMC Bioinformatics. 2007; 8:372.
27. Matys V, Fricke E, Geffers R, Gossling E, Haubrock M, Hehl R, et al. TRANSFAC: transcriptional regulation, from patterns to profiles. Nucleic Acids Res. 2003; 31(1):374-8.
28. Khan A, Fornes O, Stigliani A, Gheorghe M, Castro-Mondragon JA, van der Lee R, et al. JASPAR 2018: update of the open-access database of transcription factor binding profiles and its web framework. Nucleic Acids Res. 2018; 46(D1):D1284.
29. Huang C, Pollock CA, and Chen XM. KCa3.1: a new player in progressive kidney disease. Curr Opin Nephrol Hypertens. 2015; 24(1):61-6.
30. Huang C, Shen S, Ma Q, Chen J, Gill A, Pollock CA, et al. Blockade of KCa3.1 ameliorates renal fibrosis through the TGF-betal/Smad pathway in diabetic mice. Diabetes. 2013; 62(8):2923-34.
31. Kim M, Kim T, Johnson RL, and Lim DS. Transcriptional co-repressor function of the hippo pathway transducers YAP and TAZ. Cell Rep. 2015; 11(2):270-82.
32. Brown BM, Pressley B, and Wulff H. KCa3.1 Channel Modulators as Potential Therapeutic Compounds for Glioblastoma. Curr Neuropharmacol. 2018; 16(5):618-26.
33. Schumacher MA, Rivard AF, Bachinger HP, and Adelman JP. Structure of the gating domain of a Ca2+-activated K+ channel complexed with Ca2+/calmodulin. Nature. 2001; 410(6832):1120-4.
34. Nam YW, Orfali R, Liu T, Yu K, Cui M, Wulff H, et al. Structural insights into the potency of SK channel positive modulators. Sci Rep. 2017; 7(1):17178.
35. Mundel P, Reiser J, Zuniga Mejia Borja A, Pavenstadt H, Davidson GR, Kriz W, et al. Rearrangements of the cytoskeleton and cell contacts induce process formation during differentiation of conditionally immortalized mouse podocyte cell lines. Exp Cell Res. 1997; 236(1):248-58.

1. A compound of Formula I: 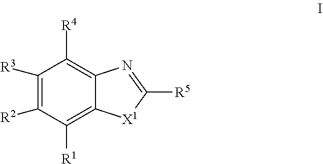
or a pharmaceutically acceptable salt thereof, wherein
X¹ is selected from N, O, and S;
R¹, R², R³, and R⁴ are each independently selected from hydrogen, halogen, CN, C(═O)N(R⁶)₂, C(═O)OR⁶, (C₃-C₁₀)carbocycle, heterocycle, and B(OH)₂;
R⁵ is selected from (C₃-C₁₀)carbocycle and heterocycle, wherein each is optionally substituted with one or more of halogen, CN, C(═O)N (R⁶)₂, C(═O)OR⁶, (C₃-C₁₀)carbocycle, heterocycle, or B(OH)₂; and
R⁶ in each occurrence is independently selected from hydrogen and (C₁-C₆)alkyl.

2. The compound of claim 1, or a pharmaceutically acceptable salt thereof, wherein
R⁵ is 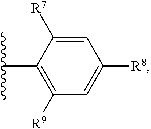
and [symbol] is the point of attachment; and
R⁷, R⁸, and R⁹ are each independently selected from hydrogen, halogen, CN, C(═O)N(R⁶)₂, C(═O)OR⁶, (C₃-C₁₀)carbocycle, heterocycle, and B(OH)₂, wherein the heterocycle is optionally substituted with one or more of (C₁-C₆)alkyl, halogen, CN, C(═O)N(R⁶)₂, C(═O)OR⁶, (C₃-C₁₀)carbocycle, heterocycle, or B(OH)₂.

3. The compound of claim 2, or a pharmaceutically acceptable salt thereof, with a structure selected from:

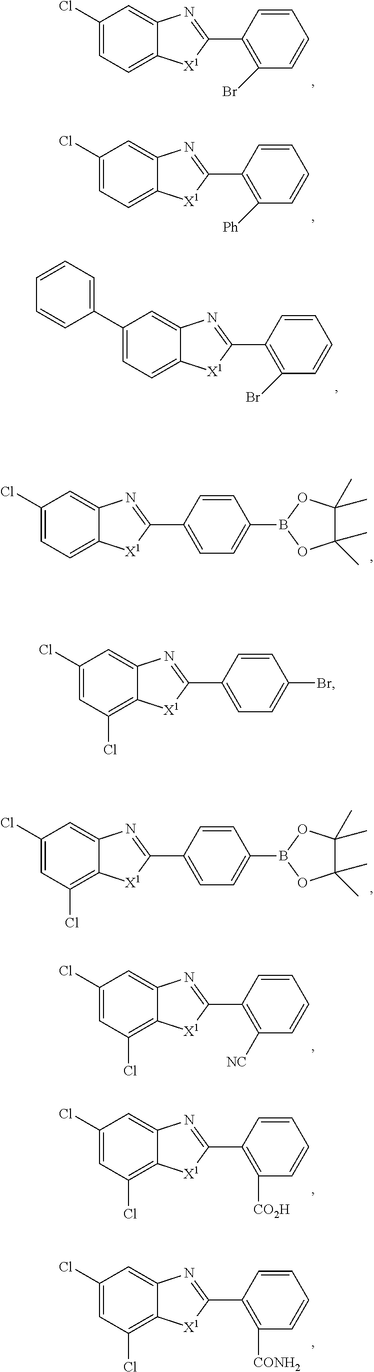

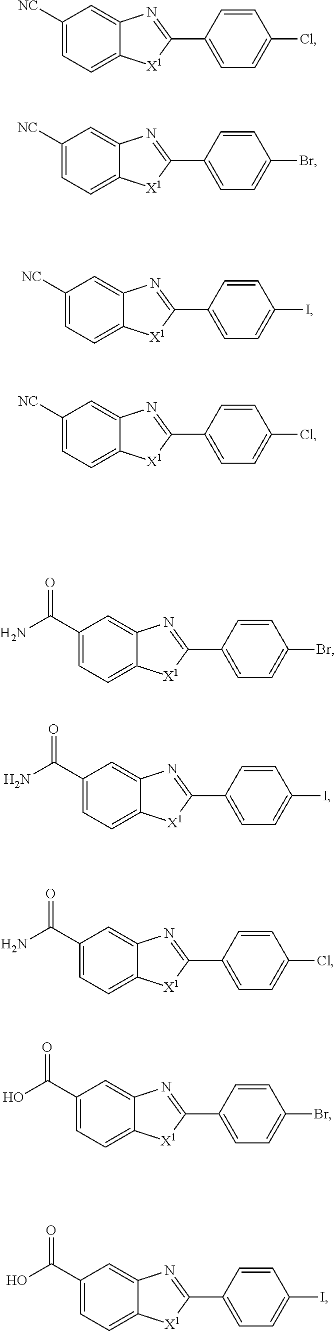

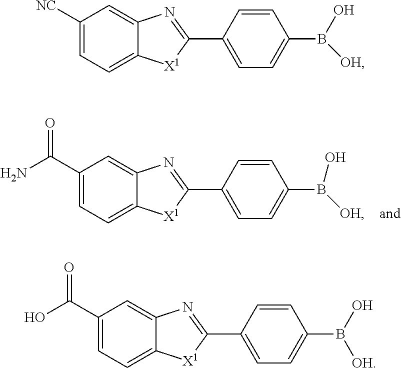

4. The compound of claim 1, or a pharmaceutically acceptable salt thereof, wherein R⁵ is a heterocycle, wherein each is optionally substituted with one or more of halogen, CN, C(═O)N(R⁶)₂, C(═O)OR⁶, (C₃-C₁₀)carbocycle, heterocycle, or B(OH)₂.

5. The compound of claim 1, or a pharmaceutically acceptable salt thereof, wherein R⁵ is selected from pyridine, pyrimidine, pyrrole, thiophene, furan, and oxadiazol, wherein each is optionally substituted with one or more of halogen, CN, ) C(═O)N(R⁶)₂, C(═O)OR⁶, (C₃-C₁₀)carbocycle, heterocycle, or B(OH)₂.

6. The compound of claim 5, or a pharmaceutically acceptable salt thereof, with a structure selected from:

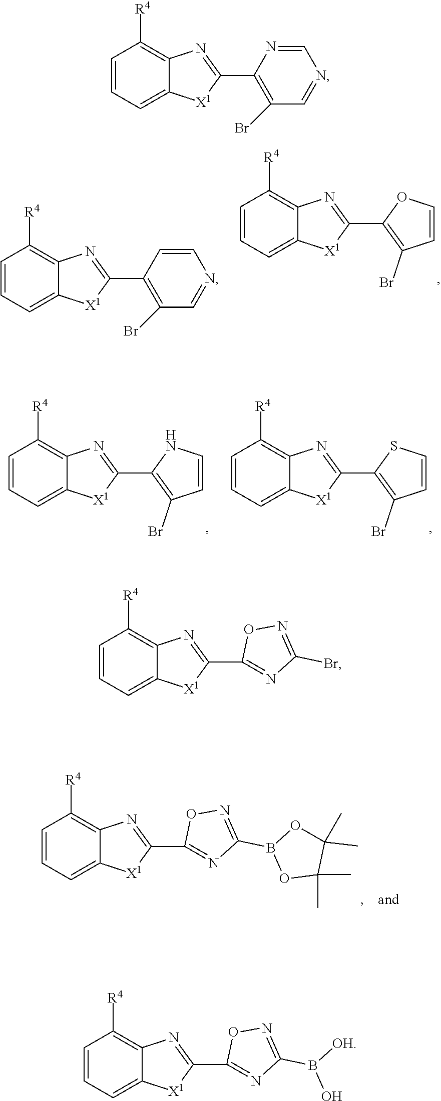

7. The compound of claim 1, or a pharmaceutically acceptable salt thereof, with a structure selected from:

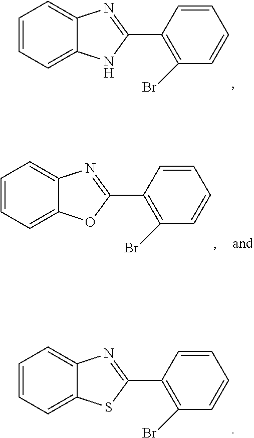 [SMILES: Brc1ccccc1-c1nc2ccccc2[nH]1 ; Brc1ccccc1-c1nc2ccccc2o1 ; Brc1ccccc1-c1nc2ccccc2s1]

8. A compound of Formula II: 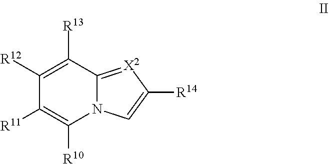
or a pharmaceutically acceptable salt thereof, wherein
X² is selected from N, O, and S;
R¹⁰, R¹¹, R¹², and R¹³ are each independently selected from hydrogen, halogen, CN, C(═O)N(R¹⁵)₂, C(═O)OR¹⁵, C(═O)R¹⁵, —N(R¹⁵)₂, (C₁-C₆)alkoxy, (C₁-C₆)thiaalkyl, (C₁-C₆)oxaalkyl, oxo(C₁-C₆)azaalkene, (C₃-C₁₀)carbocycle, heterocycle, and B(OH)₂, wherein said heterocycle is optionally substituted with one or more of —N(R¹⁵)₂, wherein R¹⁵ in each occurrence is independently selected from hydrogen and (C₁-C₆)alkyl; and
R¹⁴ is selected from (C₃-C₁₀)carbocycle and heterocycle, wherein each is optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆) azaalkene, —N(R¹⁵)₂, (C₃-C₁₀)carbocycle, heterocycle, B(OH)₂, and —N(R¹⁵)₂.

9. The compound of claim 8, or a pharmaceutically acceptable salt thereof, wherein at least one of R¹⁰, R¹¹, R¹², and R¹³ is heterocycle optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆) azaalkene, —N(R¹⁵)₂, (C₃-C₁₀)carbocycle, heterocycle, B(OH)₂, and —N(R¹⁵)₂.

10. The compound of claim 8, or a pharmaceutically acceptable salt thereof, wherein at least one of R¹⁰, R¹¹, R¹², and R¹³ is pyridine, pyrimidine, pyrrole, thiophene, furan, oxadiazole, and thiadiazole, wherein each is optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B (OH)₂, and —N(R¹⁵)₂.

11. The compound of claim 8, or a pharmaceutically acceptable salt thereof, wherein
R¹⁴ is 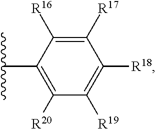
and [symbol] is the point of attachment; and
R¹⁶, R¹⁷, R¹⁸, R¹⁹, and R²⁰ are each independently selected from hydrogen, halogen, CN, C(═O)N(R¹⁵)₂, C(═O)OR¹⁵, C(═O)R¹⁵, —N(R¹⁵)₂, (C₁-C₆)alkoxy, (C₁-C₆)thiaalkyl, (C₁-C₆)oxaalkyl, oxo(C₁-C₆)azaalkene, (C₃-C₁₀)carbocycle, heterocycle, and B(OH)₂, wherein said heterocycle is optionally substituted with one or more of —N(R¹⁵)₂.

12. The compound of claim 8, or a pharmaceutically acceptable salt thereof, wherein R¹² and R¹⁴ are each independently selected from heterocycle optionally substituted with one or more of halogen, CN, C(═O)N(R¹⁵)₂, C(═O)OR¹⁵, C(═O)R¹⁵, (C₁-C₆)alkoxy, (C₁-C₆)thiaalkyl, (C₁-C₆)oxaalkyl, oxo(C₁-C₆)azaalkene, —N(R¹⁵)₂, (C₃-C₁₀)carbocycle, heterocycle, B(OH)₂, and —N(R¹⁵)₂.

13. The compound of claim 8, or a pharmaceutically acceptable salt thereof, wherein R¹⁴ is phenyl.

14. The compound of claim 8, or a pharmaceutically acceptable salt thereof, with a structure selected from: 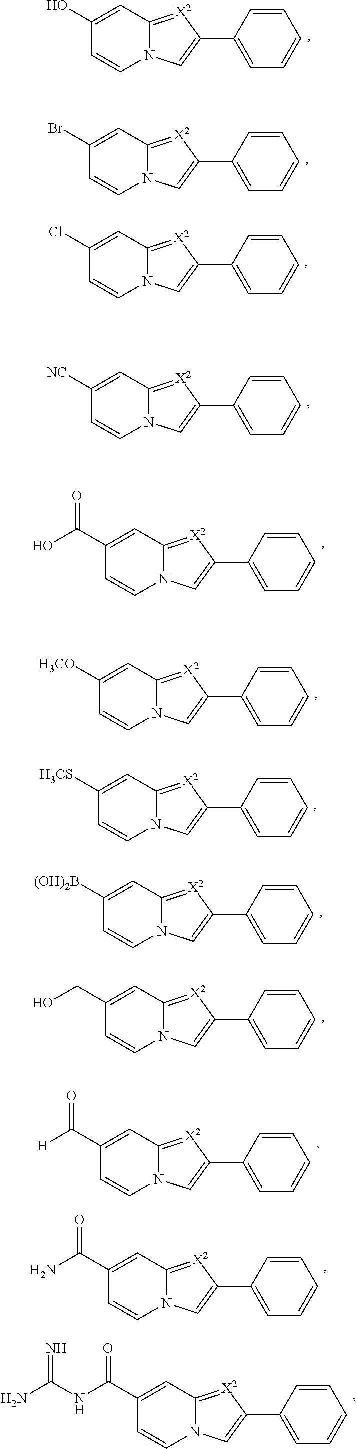 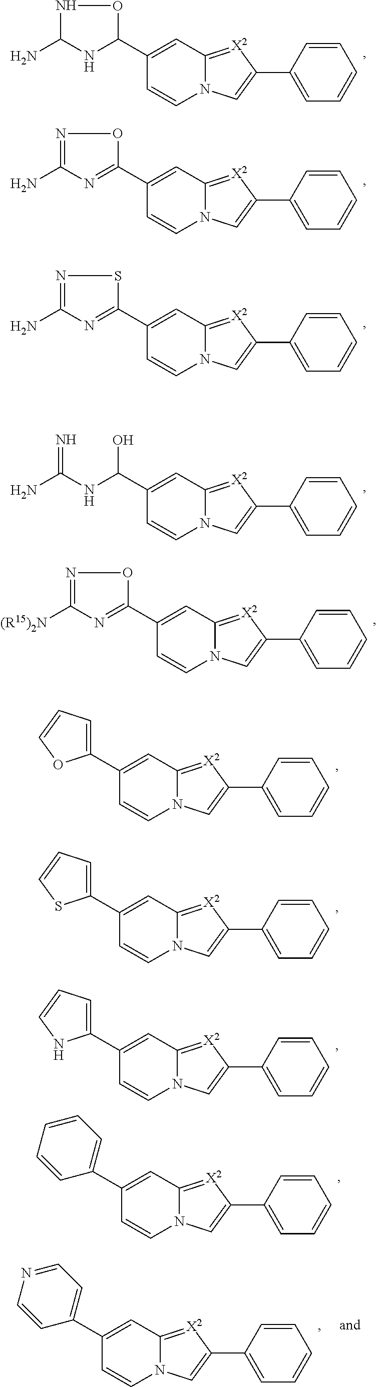 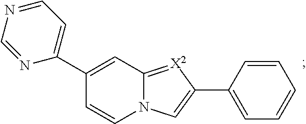
wherein X² and R¹⁵ are defined as in claim 8.

15. The compound of claim 8, or a pharmaceutically acceptable salt thereof, with a structure selected from:

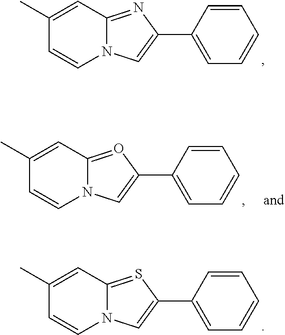

16. A pharmaceutical composition comprising a compound of claim 1 and a pharmaceutically acceptable excipient.

17. A method of treatment, comprising administering a compound of claim 1 to a subject wherein the subject suffers from kidney disease or is at risk of kidney disease.

18. A method of treatment, comprising administering the pharmaceutical composition of claim 16 to a subject wherein the subject suffers from kidney disease or is at risk of kidney disease.

19-24. (canceled)

25. A pharmaceutical composition comprising a compound of claim 8 and a pharmaceutically acceptable excipient.

26. A method of treatment, comprising administering a compound of claim 8 to a subject wherein the subject suffers from kidney disease or is at risk of kidney disease.

27-30. (canceled)
